(19) United States
(12) Patent Application Publication
(10) Pub. No.: US 2024/0139357 A1
(43) Pub. Date: May 2, 2024

(54) PASTEURIZATION OF ARCHITECTURAL COMPOSITIONS WITH ELEVATED HEAT AND METHODS THEREFOR

(71) Applicant: Columbia Insurance Company, Omaha, NE (US)

(72) Inventor: Richard Geurtsen, Robbinsville, NJ (US)

(21) Appl. No.: 18/546,840

(22) PCT Filed: Mar. 10, 2022
(86) PCT No.: PCT/US2022/019790

§ 371 Date: Aug. 17, 2023

Related U.S. Application Data
(60) Provisional application No. 63/159,199, filed on Mar. 10, 2021.

(51) Int. Cl. A61L 2/04

(52) U.S. Cl. CPC A61L 2/04; A61L 2202/18

(57) ABSTRACT

Disclosed herein are methods for pasteurizing or sterilizing architectural coating compositions using elevated heat dynamically with or without pressure.

### FIELD OF THE INVENTION

This invention generally relates to architectural coatings, including but not limited to paints and stains, that have been pasteurized or sterilized by elevated heat to remove or sufficiently reduce the level of bacteria, fungi, yeasts, and/or other biological agents in architectural coatings and to methods for pasteurizing or sterilizing same. The present invention also relates to heating architectural coatings to a temperature and duration that is sufficient to pasteurize and/or sterilize while maintaining the architectural coatings' physical and performance properties, including viscosity, to coat surfaces of buildings and abodes.

### BACKGROUND OF THE INVENTION

Due to environmental and health concerns, there has been a movement toward reducing the amount of volatile organic compounds (VOCs) in paints, stains, and other architectural coating compositions, which evaporate into the environment upon paint film formation. Additives to paints that facilitate or impart desirable paint properties, such as better film coalescence, better resistance to blocking, better film durability, better physical and chemical scrub resistance, and tougher coatings, among others, also contain VOCs. The evaporation of VOCs often results in undesirable aromas, and exposure to such fumes, particularly in areas that are not well ventilated, remains a health concern. Thus, less volatile or non-volatile additives, as well as colorants, that impart comparable (or superior) properties to the paints have been used to replace higher VOC additives. The quest for low VOC paints or a better “green paint” is discussed in a New York Times newspaper article entitled “The Promise of Green Paint” (Kershaw, Sarah, The New York Times, May 15, 2008, p. F6), which is incorporated herein by reference in its entirety.

The reduction of VOC in paints, stains and other architectural coatings and in additives, however, has produced environmentally friendly paints that are more inviting to bacteria, algae, yeasts, fungi and other biological agents that thrive in an aqueous environment. These biological agents grow and die in paint cans and containers, and often impart an unpleasant odor and render paints unusable for its intended purpose, and can cause viscosity loss, discoloration, gassing, frothing, sedimentation and pH changes. Biological agents also present potential health issues. Certain biological agents, such as algae and molds, may grow on dried paint films covering walls or other substrates.

Biocides have been used in aqueous paints or stains to control biological agents inside cans and containers. Some of the biocides may remain on the dried paint film to control algae and molds. However, there is a desire to minimize the level of biocides in aqueous paints/stains or dried paint/stain films while preventing the unimpeded growth of biological agents.

Pasteurization of paints and stains has been attempted. U.S. Pat. No. 5,529,749 to Rinno et al. teaches using dielectric heating, specifically microwaves, to reduce the microbe count in paint compositions and latex resin dispersions. Rinno also teaches that sterilization of acrylic or vinyl latex resin dispersions by direct heating to 100° C. for 1 minute or 121° C. for 1 and 3 minutes in an autoclave produces coagulations or additional cross-linking in the latex resin dispersions. Direct heating to 80° C. for 5 minutes did not cause coagulations but also did not sufficiently reduce the microbe count. The initial microbe count using CSA agar for bacteria was 1×10⁶ CFU/ml and after direct heating to 80° C. for 5 minutes was reduced only to 1×10⁴. Similarly, the initial microbe count using SDA agar for yeast was 1×10⁵ CFU/ml was reduced only to 1×10⁴. Rinno indicates that a desirable level of microbe is 1×10³ or less. Rinno does not indicate the volume of sample dispersions being heated in the autoclave or the internal temperature of the samples, and does not disclose the size of the autoclave.

Rinno also teaches that sterilization of paint compositions by gamma rays produces hydrogen peroxide and hydroxyl radicals and causes premature cross-linking of the polymer in the paints. Rinno concludes that direct heating and gamma rays are unsuitable for industrial sterilization of paint compositions.

Rinno prefers a dielectric heating, i.e., microwaving, to sterilize latex resin dispersions and paints allegedly because microwave heating does not produce coagulation or additional cross-linking. The microwave operation can be conducted in batch mode with 10-gram samples in Teflon containers and in a continuous mode with the sample being pumped through the microwave chamber. The microwave operation can be conducted under a pressure of up to 10 bars to control foaming or bubble formation.

Commonly owned U.S. Pat. No. 10,639,386 to Sheerin et al. contradicts the conclusions of Rinno. Sheerin through experimentation teaches that successful pasteurization or sterilization of paint and stain compositions can take place at significantly lower temperatures, e.g., from about 49° C. to about 72° C. (120° F. to 162° F.), from at least 120 minutes to at least 2 minutes, respectively. Sheerin also through experimentation teaches that gamma rays can successfully pasteurize paint compositions at less than about 15 kGys without further polymerization or cross-linking of the latex polymers in paints. Bacteria and molds were reduced to less than 1×10³ or no growth.

The following biological agents can be found in paints:
i. Bacteria: Pseudomonas species, including Pseudomonas aeruginosa; gram negative rod bacteria; Enterobacter aerogenes; Sphingomonas paucimobilis; other gram positive and gram-negative species etc.
ii. Yeasts: Candida lambica and Yarrowia lipolytica, etc.
iii. Fungi (molds): Aspergillus species, Acremonium species, Geotrichum species and Penicillium species, etc.

In some of the examples or experiments discussed in Sheerin, an inoculum comprising the above listed biological agents was introduced into paints or paint containers and the biological agents were allowed to grow. Thereafter, the paints were pasteurized by heat or gamma rays, and the paints were re-tested to determine the residual concentration of biological agents (if any) and whether the paints remained functional. In other examples or experiments, commercial paints with biocides that were overwhelmed by one or more known biological agents were pasteurized and re-tested to determine whether contaminated paints could be returned to the commercial conditions and be suitable for sale.

Pseudomonas aeruginosa or P. aeruginosa was found in some contaminated paints. This bacterium is commonly found in wet and warm environments, such as swimming pools and hot tubs. It has been reported by researchers from schools of medicine and public health that P. aeruginosa can grow in the range of 25° C. to 42° C. but can be killed at a temperature of 60° C., and up to 70° C. for a duration of about 30 minutes. P. aeruginosa does not grow, but does not die, at temperature of 10° C. up to 15° C. or 20° C. These results were reported by A. Tsuji, Y. Kaneko, K. Takahashi, M. Ogawa and S. Goto, 1982, The Effects of Temperature and pH on the Growth of Eight Enteric and Nine Glucose Non-Fermenting Species of Gram-Negative Rods, Microbiol. Immunol, Vol. 26(1), 15-24, 1982 at pp. 15-24 (Toho University School of Medicine, Department of Microbiology) (hereinafter “Tsuji”), which is incorporated herein by reference in its entirety.

Tsuji also reported the effects of heat on the following bacteria.

TABLE 1

| | | Survival | Growth | Peak |
|---|---|---|---|---|
| | Bacteria Species | T(° C.)^{‡} | T(° C.)† | T(° C.) |
| 1 | E. coli | 10-50 | 18-47 | 40 |
| 2 | K. pneumoniae | 10-50 | 16-48 | 36 |
| 3 | S. marcescens | 10-50 | 19-41 | 35 |
| 4 | P. aeruginosa | 10-50 | 25-42 | 37 |
| 5 | P. cepacia | 10-50 | 28-37 | 34 |
| 6 | P. fluorescens | 10-40¹ | 25-32 | 30 |
| 7 | P. maltophilia | 10-50 | 22-39 | 34 |
| 8 | A. xylosoxidans | 10-40² | 28-37 | 35 |
| 9 | A. calcoaceticus | 10-50 | 20-45 | 38 |
| 10 | A. faecalis | 10-40² | 28-37 | 36 |
| 11 | F. meningosepticum | 10-50 | 24-37 | 33 |
| 12 | Moraxella | 10-40³ | 23-35 | 30 |
| 13 | P. mirabilis | | 23-44 | 37 |
| 14 | P. vulgaris | | 22-41 | 37 |
| 15 | P. morganii | | 23-42 | 36 |
| 16 | P. rettgeri | | 24-43 | 36 |
| 17 | P. inconstans | | 22-45 | 38 |

(E. is the genus Escherichia; K. is the genus Klebsiella; S. is the genus Serratia; P. is the genus Pseudomonas; A. is the genus Acinebacter; F. is the genus Flavobacterium.)
^{‡}= survive for at least 6 hours; survival tests conducted at 10-70° C. at increments of 10° C.
¹= survive at 50° C. for about 1 hour.
²= survive at 50° C. for about 4 hours.
³= survive at 50° C. for about 2 hours.
†= growth tests conducted at 10-50° C. and the time for growth from 10² cells/ml initial concentrations to 10⁷ cells/ml was noted; bacteria growths were observed for 48 hours.

All of the tested bacteria were eradicated at temperature of 60° C. or 70° C. for duration of 30 minutes. No bacteria survived at 60° C. for more than 2 hours. None survived more than 30 minutes at 70° C.

All of the tested bacteria survived at 10° C. but did not grow. Otherwise, they grow at the reported temperature ranges, and the optimal growth, at the reported peak temperatures.

Tsuji also reported that a pH from 6.4 to 8.2 has little effect on the growth rate of these bacteria. However, S. Bricha, K. Ounine S. Oulkheir, N. E. El Haloul and B. Attarassi, 1994, Heat Resistance of Pseudomonas aeruginosa in Preparations at the Base of Cucumber, Tomato and Lettuce as Affected by pH and Sodium Chloride, ISPROMS ISSN: 1994-5108, WJBR Vol. 3, Issue 1, at 1-8 (Ibn Tofail University, Morocco) (hereinafter “Bricha”), reported that at pH of about 2 a strain of P. aeruginosa's heat resistance is diminished at temperature of 63° C., but the heat resistance of P. aeruginosa at pH of 4.5 and 6 is about the same. Bricha also reported that at low pH sodium chloride salt at 2-6% may protect the bacteria. Bricha is incorporated herein by reference in its entirety.

Yeast cells begin to die at temperature greater than 50° C. and most would die at temperatures from about 55° C. to about 60° C. It is well known to bakers that yeasts when added to water that is too warm are killed and the dough won't rise. At temperatures of 10° C. or lower, yeasts would not grow. Yeasts grow in the temperature range from about 27° C. to about 32° C. depending on the species. Hence, yeasts have a similar dormant-growth-death temperature profile as the bacteria discussed above. Hence, yeasts can be eradicated and/or controlled by the heat method including storage and transportation, as described in Sheerin.

Molds including mildew, fungi, and common molds exist in the same temperature range (and relative humidity) that supports human life. Hence, mold and mold spores are ubiquitous in our environment. Molds can grow in temperatures between 4° C. and 38° C. (40°−100° F.). Below 4° C., molds are in a dormant state and are revived when the temperature warms up with the proper relative humidity. Some molds survive a temperature as high as 38° C. or higher. The dormant-growth-death temperature regime for several molds and spores has been reported, as shown below. See http://www.thermapure.com/environmental-services/mold/.

TABLE 2

| Mold species | Lethal T(° C.) | Duration (min) |
|---|---|---|
| Alternarie altermata | 63 | 25 |
| Aspergillus fumigatus | 65 | 30 |
| Aspergillus niger | 63 | 25 |
| Chaetomium globosum | 57 | 10 |
| Cladosporium herbarum | 50 | 10 |
| Stachybotrys chartarum | 60 | 30 |

While some lethal temperatures are slightly higher than 60° C. for some of the mold discussed above, the time duration to kill is significantly shorter. At 60° C. but for longer time duration, most molds can be killed. Hence, molds can be eradicated and/or controlled by the same heating method described in Sheerin.

As taught by Rinno and Sheerin, which are incorporated herein by reference in their entireties, pasteurization or sterilization of paint and stain compositions by various methods are unpredictable. Detailed analysis and experimentation are necessary to determine the efficacy of any pasteurization technique. Sheerin teaches that paints and other architectural compositions can be pasteurized at relatively low level of heat and relatively longer hold time. Rinno discourages all forms of heat pasteurization of latex resin dispersions due to the formation of coagulations and/or additional cross-linking at temperatures of 100° C. and 121° C., and high residual level of microbes at 80° C. However, the teachings of Rinno with regard to heat pasteurization is limited to the heating of stationary samples of latex resin dispersion of unknown size/mass to an unknown internal temperature in an autoclave of unknown size. The teachings of Rinno are also contradicted by those in Sheerin.

Hence, there remains a need for additional sterilization or pasteurization techniques of architectural compositions such as paints and stains.

### SUMMARY OF THE INVENTION

Hence, the invention relates to a method for pasteurizing or sterilizing paints with heat with or without pressure to kill biological agents that may have been introduced into the paints.

An embodiment of the present invention relates to a method for pasteurizing or sterilizing an architectural coating composition comprising the steps of
(i) providing or optionally preparing the architectural coating composition;
(ii) applying heat from a heat source to said architectural coating composition to pasteurize or sterilize same by heating said architectural coating composition to an internal temperature range of at least about 100° C., preferably at least about 121° C. or preferably at least about 131° C. and dynamically moving said architectural composition through the heat source for any time duration;
(iii) storing the pasteurized architectural coating composition in containers.

Preferably, step (ii) comprises a continuous heating process.

Preferably, one of the Stormer or ICI viscosity measurements changes from preheated to post-heated is less than 10% and preferably less than about 7.5%, preferably less than about 5%, more preferably less than about 2.5%.

In one embodiment, the time duration is from about 1 minute or less to about 5 minutes or less. In another embodiment, the time duration is about 15 seconds or less, and preferably 10 seconds or less, more preferably 5 seconds of less and more preferably 2.5 seconds or less. Preferably, the pasteurization or sterilization is a flash process.

Preferably, the inventive method further comprises the step of cooling the architectural composition. The step of cooling the architectural composition may occur after step (ii) and optionally before step (iii).

The architectural coating composition preferably flows through a continuous piping through the heat source. Preferably, the continuous piping comprises heat transferring fins.

Alternatively, step (iii) occurs prior to step (ii). An example of this alternative embodiment of the present invention is a method for pasteurizing or sterilizing an architectural composition comprising the steps of
(i) providing or optionally preparing the architectural coating composition;
(ii) storing the pasteurized architectural coating composition in containers;
(iii) (a) applying heat from a heat source at about 100° C. or higher to said architectural coating composition to pasteurize same and dynamically moving said architectural composition through the heat source for a minimum time period from about 30 minutes to about 50 minutes;
   OR
(b) applying heat to said architectural coating composition to pasteurize same by heating said architectural coating composition to an internal temperature range from about 60° C. to about 92.5° C. and dynamically moving said architectural composition through the heat source for a time duration range from at least about 50 minutes to at least about 2 minutes.

Preferably, the method includes a step of cooling the architectural coating composition after the heating step (iii)(a) or (iii)(b). Preferably, in the heating step (iii)(a) or (iii)(b) a rotary sterilizer-cooler is applying heat to the architectural coating composition. Preferably, the internal temperature range in step (iii)(b) may increase or decrease by any increments of 2.5° C. between the low end and the high end. Preferably, the time duration range in step (iii)(b) may decrease or increase by any increments of 2.5 minutes between the longer end and the shorter end.

The containers include #10 cans or 1-gallon paint cans, among other containers including common paint containers, such as 5-gallon, pint and sample containers.

Other embodiments are described below.

### BRIEF DESCRIPTION OF THE DRAWINGS

In the accompanying drawings, which form a part of the specification and are to be read in conjunction therewith and in which like reference numerals are used to indicate like parts in the various views:

FIG. 1A (conventional) is a perspective view of a typical rotary sterilizer shown in U.S. Pat. No. 7,775,155; FIG. 1B (conventional) is an enlargement of portion B of FIG. 1A.

FIG. 2A shows the internal temperatures of the paint cans. FIG. 2B shows the temperatures as measured by the thermocouples within the laboratory sterilizer, but outside the paint cans being treated.

FIGS. 3A-C are the flow curves of the three commercial paints in Experiment 1 with a laboratory retort on a log-log scale of the viscosity of the paint samples (Y-axis, Pascal seconds, or Pa-s) as a function of the applied shear rate or shear stress (X-axis, second⁻¹).

FIGS. 4A-C are the flow curves of the three commercial paints in Experiment 2.

FIGS. 5A and 5B are schematic diagrams of exemplary sterilization apparatus operating in a continuous mode. Although a rotary sterilizer is generally used for the in-can sterilization of food products, we investigated its use for the in-can pasteurization of architectural compositions.

FIGS. 6A and 6B are the flow curves of the commercial paint in Experiment 3 at 132° C. and 140° C.

### DETAILED DESCRIPTION OF THE PREFERRED EMBODIMENTS

As used herein, paints or stains include aqueous or water-based paint or stain compositions and other architectural compositions, which can be pasteurized or sterilized. Paint films mean paints or stains that have been applied on a surface or substrate, and are dried or the latex particles in the paints and stains have coalesced or cross-linked to form the films. Architectural compositions also include materials understood in the art as architectural compositions, such as adhesives, caulking, asphalts, etc.

The pasteurization or sterilization techniques of the present invention include a dynamic elevated heating (hereinafter “DEH”) process utilizing a rotary cooker-cooler (also known as a rotary sterilizer-cooler), a hot fill-hold process, an aseptic technique, and a continuous sterilization technique preferably utilizing DEH. These processes and techniques are applicable to all architectural compositions. The technique utilizing the rotary cooker-cooler and the hold-fill-hold technique are pasteurization techniques, and the aseptic technique is a sterilization technique. The difference between pasteurization and sterilization is the induction temperature. Pasteurization generally operates up to 100° C. to inactivate vegetative microbes, while sterilization operates above 100° C. to inactivate spores or spore-forming pathogens.

As discussed above, water-based latex architectural coatings, such as paints, stains, other household, and industrial coatings, have become more environmentally friendly. This means that modern day architectural coatings have less VOCs in them and have additives and colorants that also have low VOCs. The reduction in VOCs have rendered the architectural coatings more inviting to biological agents, such as bacteria and fungi in the water phase and algae and certain fungi, e.g., molds, in the dried film phase. One solution is to add biocides to the architectural coatings at the latex formation stage, at the pigment dispersion stage, where pigments are dispersed with surfactants, dispersants and water, and/or the let-down stage, where the aqueous latex, the pigment dispersion and additives are combined. The paints are then placed in cans and containers for storage and shipping. Colorants, which may contain their own biocides, are added later at the retail stores to achieve the paint colors that the consumers purchased.

While biocides are useful to preserve paints and other architectural coatings and are useful in the dried paint film to help prevent the growth of biological agents, some environmentally conscientious consumers have expressed a desire for biocide-free or biocide-reduced paints. However, without biocide or with reduced biocides, biological agents could thrive in aqueous paints and stains or dried films.

### Experiment 1. Pasteurization with a Rotary Sterilizer-Cooler

In accordance with one embodiment of the present invention, paints, stains and other architectural compositions can be pasteurized by the DEH process. DEH includes but is not limited to a pasteurization process that heat the architectural composition up to an internal temperature up to, but not exceeding, 100° C. DEH also includes sterilization at temperatures exceeding 100° C. and includes batch and continuous processes. Preferably the internal temperature is in the range of about 60-about 80° C. The pasteurization takes place through heat transfer—including heat conduction, heat convection and/or heat radiation as used in all embodiments/experiments described in the present invention—from an applied hot air, hot water, or steam preferably under pressure to the architectural composition, while the architectural composition is moved, stirred, rotated, or otherwise in a non-stationary manner in sealed paint containers or pushed through continuous piping.

Three commercial paints and one biocide-free experimental paint, which was inoculated as described below, underwent such a thermal treatment process. The DEH treated paints were tested and compared to the untreated samples. The paints were stored in common #10 cans typically used to store foods such as canned tomatoes and other fruits and vegetables. These cans have typical dimensions of 6.25 inch diameter and 7 inch height, and a volume capacity from about 102 oz. to 111 oz. with an average capacity of about 109 oz. The #10 can was selected for this experiment because its volume and dimensions are close to those of a paint can (128 oz. and 6.5 inch diameter×7.5 inch height). The #10 cans were filled with paint samples leaving approximately a ½ inch head space.

The #10 cans filled with paints were pasteurized in a laboratory pressure sterilizer machine, which is a simplified version of a typical rotary sterilizer. The laboratory pressure sterilizer can supply pressurized steam, hot water, or superheated water with an air overpressure process. It can simulate hot water or saturated steam rotary processes. The cans are rotated about their own axis while the machine rotates to induce convective heating and cooling. The laboratory pressure sterilizer is designed for conducting rotary sterilizer pilot plant studies. The equipment is a simplified version, containing one rotating reel (ring) to hold sample cans, of a full-size rotary sterilizer. Steam was used to heat the samples and well or tap water was used to cool

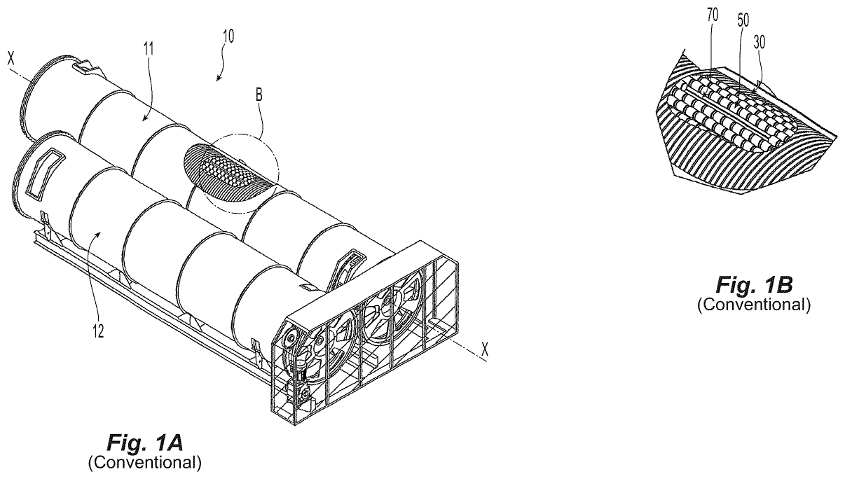

An example of a typical full-size rotary sterilizer is described in U.S. Pat. No. 7,775,155, entitled “Rotary Cooker for Use with Chamfered, Stackable Cans” to A. S. Van Rooyen and originally assigned to H.G. Molenaar & Co., and in United States Published Patent Application No. 2012/0132502 entitled “Can Transfer System” to T. L. Thring et al. These references are incorporated herein by reference in their entireties. FIGS. 1A and 1B, which are from the '155 patent, show a typical rotary sterilizer-cooler 10 having two parallel elongated chambers 11 and 12. The chambers can be in the order to ten meters or more long and two meters or more in diameter. An array of helical rails 30 guide and support cans 50 as they convey cans 50 from left to right in FIG. 1A around horizontal axis X-X. Cans 50 are propelled along helical rails 30 by brackets 70, as described in the '155 patent. First chamber 11 is typically heated and cooks or sterilizes cans 50 moving therethrough. Second chamber 12 typically cools cans 50 as they move in the opposite direction. Rotary sterilizer-cooler 10 is sized and dimensioned to receive #10 cans. The cans roll on their sides as they are conveyed through rotary sterilizer-cooler 10. Hence, the paints within these cans are dynamically moving rotationally within the can as the cans are moved translationally through rotary sterilizer-cooler 10.

A thermocouple is inserted through a lid and into the cans that contain the three commercial paint samples for this experiment. Several thermocouples are also positioned within the laboratory pressure sterilizer and outside the cans to measure the temperature of the heat applied to the cans. As discussed above, Tsuji teaches that no bacteria survives at 70° C. for more than 30 minutes. In this experiment, the laboratory pressure sterilizer's temperature was set at about 100° C. (212° F.), and the target internal temperature inside the cans was selected at about 75° C. (167° F.). It is noted that the target temperature can be set higher or lower as taught in Sheerin and Tsuji.

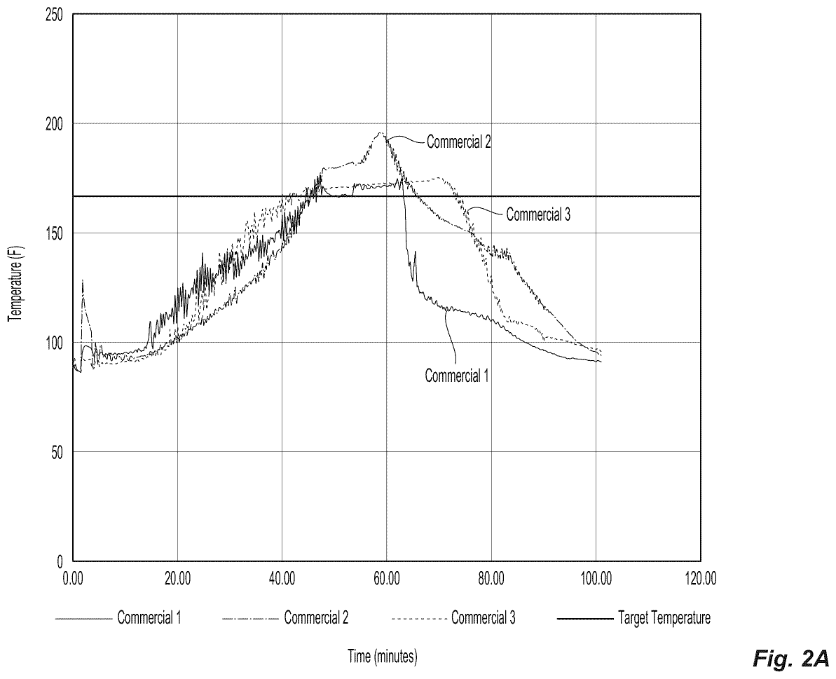

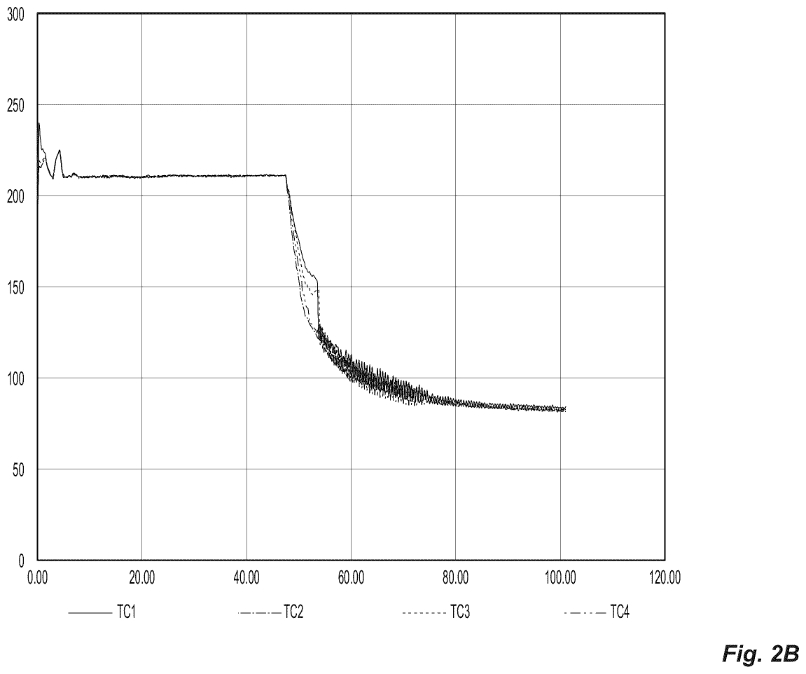

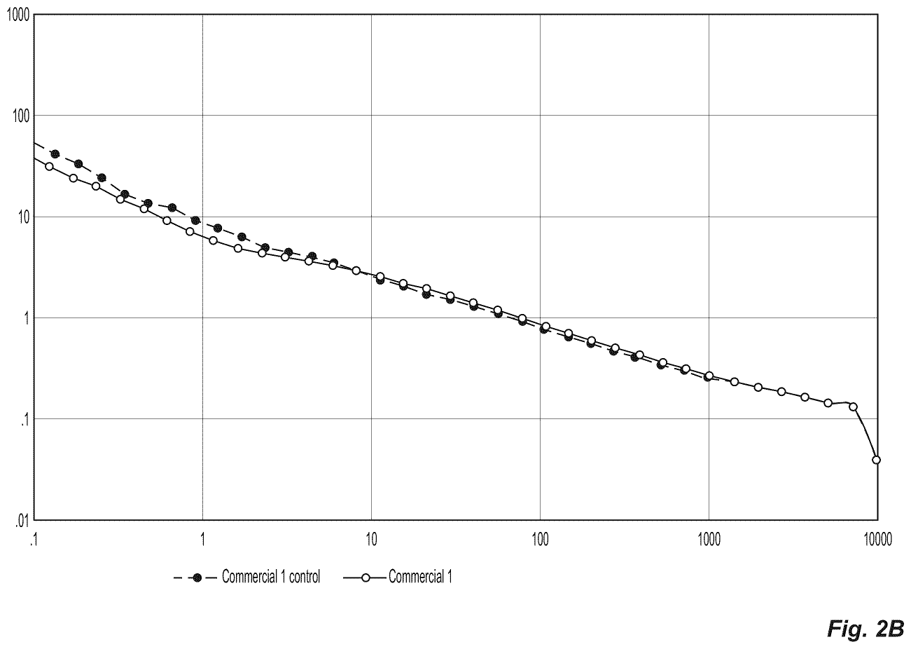

FIG. 2B shows the temperatures measured by the thermocouples within the sterilizer-cooler but outside the cans. The cans were heated by the sterilizer-cooler at 100° C. for about 50 minutes and were transferred to the cooling section, as shown by the drops in temperature beginning at about the 50-minute mark. FIG. 2A shows the internal temperatures of the paint cans measured by the internal thermocouples, which gradually increases until the cooling phase and then decreases. The data shows that for Commercial #1, which is a stain blocking white primer paint, the time duration at or above the target pasteurization temperature was 17.5 minutes (from 45.50 minute mark to 63 minute mark). For Commercial #2, which is 1-base premium interior flat paint and has the highest level of TiO₂ opacifying pigment, the time duration at or above the target pasteurization temperature was 21 minutes (from 42.25 minute mark to 66.25 minute mark). For Commercial #3, which is a 4-base premium interior semi-gloss paint and has the lowest level of opacifying pigment and the highest level of latex resin, the time duration at or above the target pasteurization temperature was 32 minutes (from 41.50 minute mark to 73.5 minute mark). The significantly longer time duration for Commercial #3 at or above the target temperature was likely caused by the low amount of opacifying pigment (TiO₂) contained in a 4-base paint. The highest internal paint temperatures were recorded at 195.78° F. (91° C.) at 58:45 and 59:15 time markers for Commercial #2 which has the highest level of opacifying pigment. Since the viscosity at different shear rates of Commercial #2 paint remains within acceptable levels, as shown below, Commercial #2 and similar paints and their latex binders can handle pasteurization temperatures up to 91° C. and higher.

In Experiment 1, the heating period extended to about 50 minutes. The data in Appendix 1 shows that the 60° C. pasteurization temperature was reached in Commercial #1 and #3 at about the 30 minute marker and 38 minute marker for Commercial #2. Hence, the heating period for this experiment can be as short as about 30 minutes. It is noted that for smaller cans and for continuous processes where paints are conducted through pipes, the heating time to reach the target internal pasteurization can be greatly reduced to the order of less than 1 minute to about 2 minutes, as discussed below.

TABLE 3 below shows the time durations and temperatures that Commercial ##1, 2 and 3 paints experienced in Example 1.

TABLE 3

| | Commercial | Commercial | Commercial |
|---|---|---|---|
| Internal Temperature | #1 (min) | #2 (min) | #3 (min) |
| 140° F. (60° C.) | 34.75 | 42.25 | 48.25 |
| 149° F. (65° C.) | 25.00 | 35.25 | 43.50 |
| 158° F. (70° C.) | 20.50 | 26.75 | 38.50 |
| 167° F. (75° C.) | 17.5 | 21.00 | 32.00 |
| 176° F. (80° C.) | 0.50 | 16.50 | * |
| 185° F. (85° C.) | * | 6.00 | * |
| 194° F. (90° C.) | * | 2.00 | * |

* did not reach temperatures

Lower internal pasteurization temperatures would require longer pasteurization time and higher internal pasteurization temperatures would require shorter pasteurization time. The time durations shown in Table 3 for a lower internal pasteurization temperature would include the time durations spent at higher internal pasteurization temperatures. In other words, for example the time duration at 65° C. or higher would include the time spent at 70° C., 75° C. and so on.

Preferably, the internal pasteurization temperature range is from about 60° C. to 92.5° C. and said range may increase or decrease by any increments of 2.5° C. from the low end to the high end. Any incremental value can serve as the low end or high end of the internal pasteurization temperature range. Minimum suitable pasteurization time duration range is from about 50 minutes at the low end of the internal pasteurization temperature range to 2 minutes at the high end of the internal pasteurization temperature range, and may decrease or increase from the longer time to the shorter time by increments of 2.5 minutes. Any incremental value can serve as the low end or the high end of the time duration range. It is noted that these time durations are the minimum time to be held at the targeted internal pasteurization temperature. As taught in Sheerin et al. and discussed in its prosecution history in the U.S. Patent and Trademark Office the time duration may extend beyond the minimum time duration, so long as the paints are not negatively affected, e.g., by changes in viscosity beyond the acceptable ranges discussed herein. For completeness, preferably the maximum suitable pasteurization time durations range from about 360 minutes at the low end of the internal pasteurization temperature range to 15 minutes at the high end of the internal pasteurization temperature range, and may decrease from the longer time to the shorter time by increments of 5 minutes. Any incremental value can serve as the low end or the high end of this time duration range.

A can containing an experimental paint that was inoculated as discussed below was also treated by the DEH process along with the three commercial paints. Another can containing the same inoculated experimental paint was kept untreated to serve as a control.

It is noted that as taught in the commonly owned Sheerin patent and the Tsuji paper the internal pasteurization temperature could be set at a lower temperature, e.g., 70° C. or 60° C. or any temperature in between, or at a higher temperature. The temperature data for Experiment 1 is attached in the Appendix.

A relevant metric for evaluating the paints, stains and other architectural compositions treated by DEH or other heat treatment or other pasteurization processes is whether the viscosity at one shear rate of the compositions changes significantly. Preferably, one of the viscosity measurements changes from pre-heat (or unheated) to post-heat treatment can be as high as 10% and preferably less than about 7.5%, preferably less than about 5%, more preferably less than about 2.5%. In other words, at least the change in one of the Stormer viscosity or ICI viscosity, which are measured at different shear rates, should be within these preferred ranges. Preferably, the viscosity measurements are averaged, Alternatively, the post-heat treatment viscosity is within the allowed viscosity range of the paint's specification. The static viscosity (in-the-paint-can viscosity) of these commercial paint samples and the treated samples was measured, as shown in Table 3 below.

TABLE 4

| | | | ICI (P) | | | |
|---|---|---|---|---|---|---|
| | Stormer (KU) | | Spec/ | | pH | |
| Paint | Spec/Lot | Actual | Measured† | Actual | Spec | actual |
| Commercial 1 | 95.0- | | primer§/ | | 8.6-9.3 | |
| control | 101.0/ | | 1.217 | | | |
| Commercial 1- | 100.3 | 99.2 | | 1.425 | | 8.9 |
| DEH | | (1.1%) | | (17.1%) | | |
| Commercial 2 | 92.0- | | 1.000- | | 8.6-8.8 | |
| control | 96.0/ | | 1.300/ | | | |
| Commercial 2- | 92.6 | 102.2 | 1.242 | 1.163‡ | | 8.3 |
| DEH | | (10.3%) | | (6.4%) | | |
| Commercial 3 | 100.0- | | 1.100- | | 8.6-9.2 | |
| control | 106/ | | 1.350/ | | | |
| Commercial 3- | 104.0 | 105.4 | 0.925 | 0.925 | | 9.1 |
| DEH | | (1.35%) | | (0%) | | |

†The measured ICI viscosity values are from Table 7, supra.
§specifications for primer paints do not typically include range for ICI viscosity.
‡within specification's allowed viscosity range.

As shown, the Stormer and ICI viscosity of the DEH treated samples are close to the viscosity in the paints' specification or lot/measured viscosity, showing that the DEH treatment does not significantly affect the viscosity of the paints, from which can be inferred that DEH treatment does not significantly affect the colloidal stability of the commercial paints. Very small amounts of skin for Commercial 1 and 3 in the order of 1 gram, were left on a fine filter when the treated commercial paints were filtered after treatment.

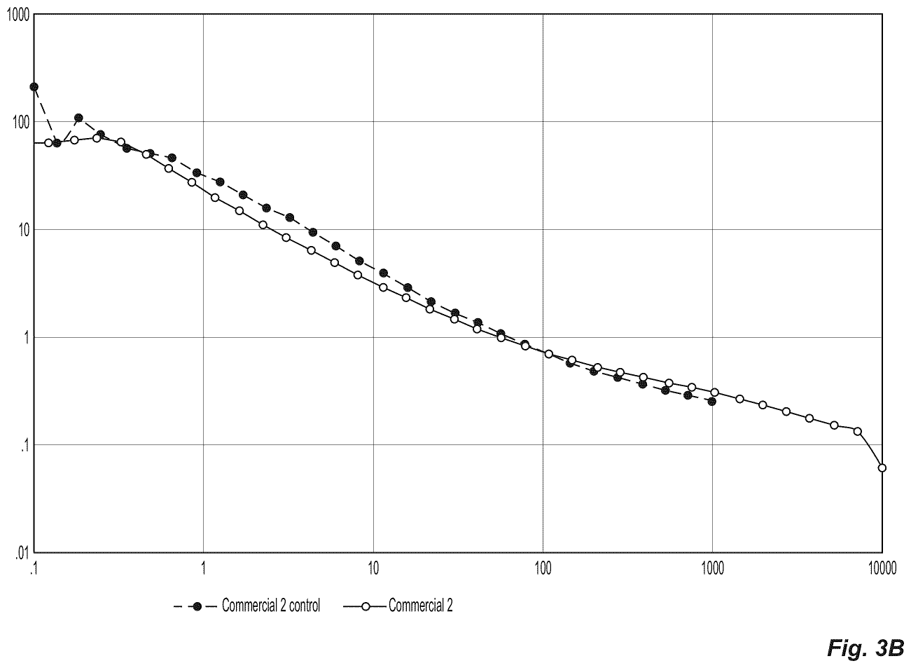

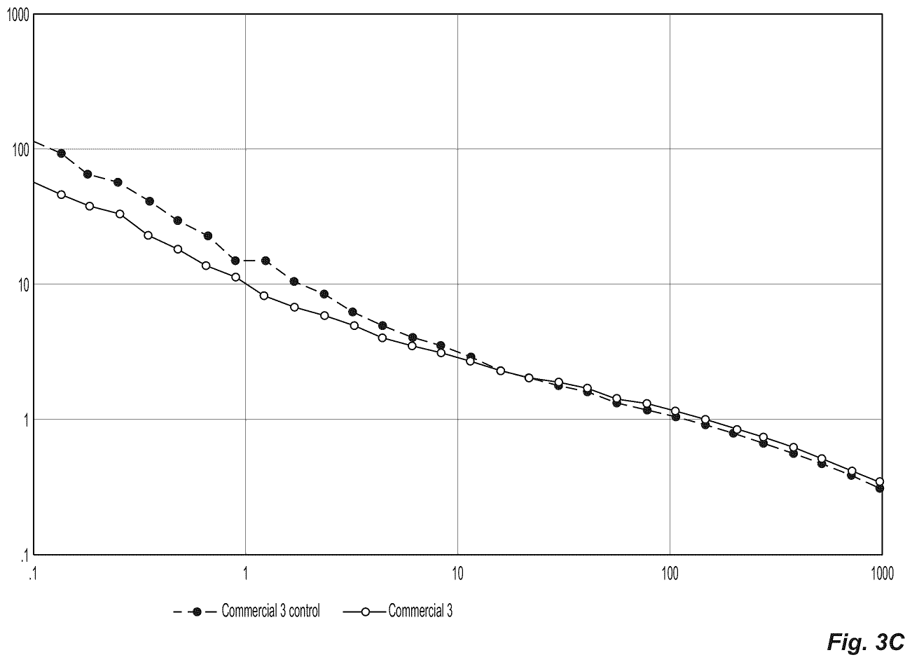

Flow curves are plotted on log-log scales of the viscosity of the paint samples (Y-axis, Pascal seconds) as a function of the applied shear rate or shear stress (X-axis, second⁻¹). Viscosity quantifies the compositions' resistance to flow. The shear rate is the change in strain over the change in time. The viscosity of the samples (vertical axis) was measured at different sheer rate (spin speed) (horizontal axis). Low spin speed mimics the stage when the paint is at substantially static conditions. At this stage, high viscosity is desirable indicating low color flow and low color separation. High spin speed mimics the stage when a user is applying the paint onto a surface, e.g., moving paint brushes or rollers. At this stage, low viscosity is desirable indicating easier application. FIGS. 3A-C show that the flow curves for all three paint samples are substantially the same between the treated samples and the untreated control samples at the higher spin speeds, which is desirable, and are close to each other at lower spin speeds. Hence, the ranges of acceptable change in ICI and Stormer viscosity between treated and untreated paints are sufficient to ensure substantially similar physical and performance properties of the architectural coatings, as illustrated by the flow curves FIGS. 3A-C.

Typical microbial species in an inoculation is discussed in Sheerin et al. The microbial species used in this experiment included the following:

TABLE 5

| | | | Mor- | Spore- |
|---|---|---|---|---|
| | | Attri- | phol- | former |
| Organism | Type | bute | ogy | (Y/N) |
| Pseudomonas aeruginosa | Gram-negative | aerobic | Rod | N |
| | bacteria | | | |
| Gram-negative rod | Gram-negative | aerobic | Rod | N |
| (grey) | bacteria | | | |
| Pseudomonas | Gram-negative | aerobic | Rod | N |
| pseudoalcaligenes | bacteria | | | |
| (in P. aeruginosa group) | | | | |
| Proteus vulgaris (nitrate- | Gram-negative | aerobic & | Rod | N |
| reducing, HS-producing) | bacteria | facultative | | |
| | | anaerobic | | |
| Brevundimonas diminuta | Gram-negative | aerobic | Rod | N |
| | bacteria | | | |
| Pseudomonas mendocina | Gram-negative | aerobic | Rod | N |
| | bacteria | | | |
| Shewanella | Gram-negative | facultative | Rod | N |
| putrefaciens* | bacteria | anaerobic | | |
| Escherichia coli | Gram-negative | facultative | Rod | N |
| | bacteria | anaerobic | | |
| Pseudomonas putida | Gram-negative | aerobic | Rod | N |
| | bacteria | | | |
| Burkholderia cepacia | Gram-negative | aerobic | Rod | N |
| | bacteria | | | |
| Staphylococcus aureus | Gram-positive | facultative | Cocci | N |
| | bacteria | anaerobic | | |
| Alcaligenes faecalis | Gram-negative | aerobic | Rod | N |
| (aka Bordetella avium) | bacteria | | | |

The biological activity results for the DEH treated, inoculated experimental paint sample versus the control, non-treated experimental paint sample are shown in Tables 6(a)-(b). Aerobic plate count (APC) is an indicator of bacterial population on a sample and is measured in “cfu/g” unit, which stands for colony forming units per gram of sample. APC assumes that each cell would form a visible colony when mixed with agar containing the appropriate nutrients. It is a generic test for organisms that grow aerobically or requiring oxygen at mesophilic or moderate temperature (25-40° C. or 77-104° F.).

The APC results in the Tables below were obtained without dilution. Of the resulting mixture, 1.0 ml was plated onto two plates. Trypticase soy agar (TSA) was poured in one plate, and sab dextrose (SAB) in the other. The TSA will grow bacteria, and the SAB will grow yeast and mold, although some gram-negative bacteria will also grow in the SAB agar.

The colonies that grew on the plates were counted.

TABLE 6(a)

| | Aerobic Plate Count (APC) | | | | | Sab |
|---|---|---|---|---|---|---|
| | | Gram+ | | Gram− | | Dextrose |
| | cfu/g | (cfu/g) | Type | (cfu/g) | Type | (cfu/g) |
| Inoculated | 2,100 | 1,500 | Cocci | 600 | Rod | >10 |
| Control | *** ENRICHMENT *** | | | | | |
| (no DEH) | E-coli | S. aureus | P. aeruginosa | Salmonella | Sab Dextrose | |
| | negative | negative | negative | Negative | No growth | |
| | Trypto Soy Agar: | | Gram+ | Type | Gram- | Type |
| | | | Yes | Rod & | Yes | rod |
| | | | (not S. aureus) | cocci | | |

TABLE 6(b)

| | Aerobic Plate Count (APC) | | | | | Sab |
|---|---|---|---|---|---|---|
| | | Gram+ | | Gram− | | Dextrose |
| | cfu/g | (cfu/g) | Type | (cfu/g) | Type | (cfu/g) |
| Inoculated | 30 | 30 | Cocci | No | N/A | <10 |
| and DEH | *** ENRICHMENT *** | | | | | |
| treated | E-coli | S. aureus | P. aeruginosa | Salmonella | Sab Dextrose | |
| | Negative | Negative | Negative | Negative | No growth | |
| | Trypto Soy Agar: | | Gram+ | Type | Gram- | Type |
| | | | Yes | Rod & | No | N/A |
| | | | (not S. aureus) | cocci | | |

The initial level of bacteria was 1,500 cfu/g gram-positive and 600 cfu/g gram-negative, and a total of 2,100 cfu/g. The after-treatment residual level was 30 cfu/g gram-positive and below-detection level of gram-negative.

Typically, the initial growth in a control sample is in the 10⁵ (level 3) to 10⁶ range (level 4), as reported in Rinno and Sheerin. In this experiment, the initial growth was at 10³ (level 2). The properties of the three commercial paints and the experimental paint are listed below. All percentages are based on weight.

TABLE 7

| | Head | Density | % | % | % | Wt. % | % |
|---|---|---|---|---|---|---|---|
| Paint | space | (lb/gal) | solids | binder | pigment | water | solvent |
| Exp. | 13.4 | 9.010 | 37.299 | 28.337 | 6.529 | 57.397 | 4.648 |
| Comm. 1 | 4.5 | 10.793 | 54.472 | 21.824 | 30.394 | 43.010 | 1.598 |
| Comm. 2 | 4.5 | 11.253 | 56.169 | 15.717 | 36.257 | 43.355 | 0.064 |
| Comm. 3 | 13.4 | 9.115 | 41.426 | 29.403 | 8.271 | 57.969 | 0.029 |

The experimental paint contains more non-exempt solvent than the commercial paints. Solvents are removed or greatly reduced before paints are commercialized. The solvent could come from one or more additives or colorants discussed above. Solvents are known historically to be hostile to microbials, as discussed above. The inoculated experimental paint was also stored for a number of days before Experiment 1 was conducted, which could have further reduced the level of biological agents. Other than the solvent level, the experimental paint is similar to Commercial paint #3, which is a 4-base paint. It is expected that the initial level of biological agents would be at the levels reported in Rinno and Sheerin had the solvent level matched those of commercial paints. It is also expected that the heat applied in Experiment 1, which produced internal paint temperatures higher than those reported in Sheerin, would reduce the microbe counts at or higher levels than shown in Sheerin.

The APC does not tell exactly what types of bacteria are present; it is a quantitative test. That is why an enrichment test was used, which is a qualitative test; 10 grams of product was added to a jar containing 90 mL of letheen broth, and the mixture was incubated for 48 hours. Next, differential media were used, wherein each is an indicator to specific bacteria by changing the color of the media, or the color of the growth on the media. Media that were used are specific for E. coli, S. aureus, P. aeruginosa, and Salmonella. A drop of broth was introduced to each medium with a loop.

The analysis showed that the reduction was 30 cfu/2100 cfu or a 2-log reduction. Preferably, the eradication is at least a 2-log (99.0%) reduction, preferably a 3-log (99.9%), a 4-log (99.99%) reduction and more preferably a 5-log (99.999%) or more reduction. Since the temperatures of the applied fluids, including gases and liquids, in this experiment are higher than those applied in Sheerin et al., the applied temperatures from Experiment 1 would have reduced microbial populations from a starting population of 10⁶ level to a 5-log reduction or more.

Hence, the experiments above show that (i) paint and stain compositions, as well as the other architectural compositions, can be treated by a rotary sterilizer pasteurization technique utilizing dynamic elevated heat (DEH) process and maintain their functionalities, and (ii) a rotary sterilizer pasteurization treatment can reduce the bacterial count in paint and stain compositions.

Experiment 1 may be summarized as a preferred method for pasteurizing an architectural coating composition comprising the steps of providing or optionally preparing the architectural coating composition;
storing the pasteurized architectural coating composition in containers;
(a) applying heat from a heat source at about 100° C. or higher to said architectural coating composition to pasteurize same and dynamically moving said architectural composition through the heat source for a minimum time period from about 30 minutes to about 50 minutes;
   OR
(b) applying heat to said architectural coating composition to pasteurize same by heating said architectural coating composition to an internal temperature range from about 60° C. to about 92.5° C. and dynamically moving said architectural composition through the heat source for a time duration range from at least about 50 minutes to at least about 2 minutes;

This method may further comprise a step of cooling the architectural coating composition after the heating step. Preferably in the heating step a rotary sterilizer-cooler is applying heat to the architectural coating composition.

The temperatures reached inside commercial paints 1, 2 and 3 are within the flash pasteurization range known in the art of food pasteurization. As taught by the International Dairy Foods Association (www.idfa.org/pasteurization), the temperatures and the time durations, which can be less than 1 second, are as follows:

| Temperature | Time | | Pasteurization Type |
|---|---|---|---|
| 63° C. (145° F.) | 30 | minutes | Vat pasteurization |
| 72° C. (161° F.) | 15 | seconds | High temperature short time |
| | | | Pasteurization (HTST) |
| 89° C. (191° F.) | 1 | second | Higher-heat shorter time |
| | | | (HHST) |
| 90° C. (194° F.) | 0.5 | second | HHST |
| 94° C. (201° F.) | 0.1 | second | HHST |
| 96° C. (204° F.) | 0.05 | second | HHST |
| 100° C. (212° F.) | 0.01 | second | HHST |
| 138° C. (280° F.) | 2 | seconds | Ultra pasteurization (UP) |

Since the internal temperature of all the paints in Experiment 1 reached 75° C., the pasteurization in Experiment 1 is at least a HTST flash pasteurization and is within the HHST flash pasteurization range. The paints tested in Experiment 1 were maintained at these flash pasteurization temperatures for much longer time periods.

Hence, Experiment 1 may be restated as a preferred method for pasteurizing an architectural coating composition comprising the steps of
providing or optionally preparing the architectural coating composition;
storing the pasteurized architectural coating composition in containers;
(a) applying heat from a heat source at about 100° C. or higher to said architectural coating composition to pasteurize same and dynamically moving said architectural composition through the heat source for any time period;
   OR
(b) applying heat to said architectural coating composition to pasteurize same by heating said architectural coating composition to an internal temperature range from about 60° C. to about 92.5° C. and dynamically moving said architectural composition through the heat source for any time period;
   wherein a change in either a Stormer viscosity or ICI viscosity from untreated to post-heat treatment is less than about 10%, preferably less than about 7.5%, preferably less than about 5%, more preferably less than about 2.5%.

The time duration can be about 15 seconds or less, and preferably 10 seconds or less, more preferably 5 seconds of less and more preferably 2.5 seconds or less.

### Experiment 2. Aseptic and Hot-Fill-Hold (HFH) Processes

In this experiment, the paints are heated to higher temperature(s) for a shorter amount of time and then removed from the heat. The paint samples are heated by saturated steam to 250° F. (121° C.) for about 5 minutes and 268° F. (131° C.) for about 1 minute in a 1-quart retort or pressure cooker (represents aseptic processing). Another set of the same paint samples was boiled in a water bath to 212° F. (100° C.) for 30 minutes (represents HFH processing, which can be regarded as an alternate pasteurization technique). All treated paints had unheated controls. All retorts for testing of aseptic processing conditions are fluidly connected to a common steam surge tank, so that all retorts received steam at the same temperature. The duration of the exposure was regulated by inlet and outlet valves on the retorts.

A quantity of about 15 g-20 g of each of the three commercial paints discussed above was sealed in a “thermal death time” (TDT) can. A TDT can is small and typically has a diameter of about 2.5 inch and is about 0.375 inch in height, so that heat can penetrate the interior of a TDT can quickly. A TDT can's maximum holding capacity is about 21 grams. Small volume and small mass of the tested paints were selected in part so that the internal temperatures of the TDT cans reaches the applied fluid temperature outside of the TDT cans faster. This mimics the temperature regimes in a continuous pasteurization process or sterilization process.

The internal temperature of TDT cans that are submerged in boiling water for 30 minutes would have reached 100° C. It is also believed that the internal temperature of the TDT cans heated for 5 minutes or 1 minute in the steamed retort would have reached a target pasteurization temperature, e.g., 75° C. (167° F.) or as taught in the literature discussed above, due to the thinness of the TDT cans. The TDT cans that were heated for 5 minutes would have reached the applied temperature of 250° F. (121° C.).

The treated samples were inspected visually, and flow curves were prepared for the treated samples and the controls. Additionally, a 2-mil thick drawdown was prepared for each sample on Leneta charts and ICI viscosity were measured. The ICI viscosity measurements, which show only minor changes from the control sample, and the drawdowns illustrated that the commercial paints' functionalities are maintained.

TABLE 8

| | ICI (P) | | | | | |
|---|---|---|---|---|---|---|
| Paint | Control | 100° C. | 121° C. | 131° C. | Visual | Drawdown |
| Comm 1 | 1.217 | 1.179 | 1.200 | 1.142 | OK^{a} | OK |
| | | (3%) | (1.4%) | (6%) | | |
| Comm 2 | 1.242 | 1.204 | 1.192 | 1.292 | OK^{b} | OK |
| | | (3%) | (4%) | (4%) | | |
| Comm 3 | 0.925 | 0.929 | 0.842 | 0.896 | OK^{a} | OK |
| | | (0.4%) | (9%) | (3%) | | |

^{a}trace of skin/solid.
^{b}some TiO₂ reacted with lining material on TDT cans.

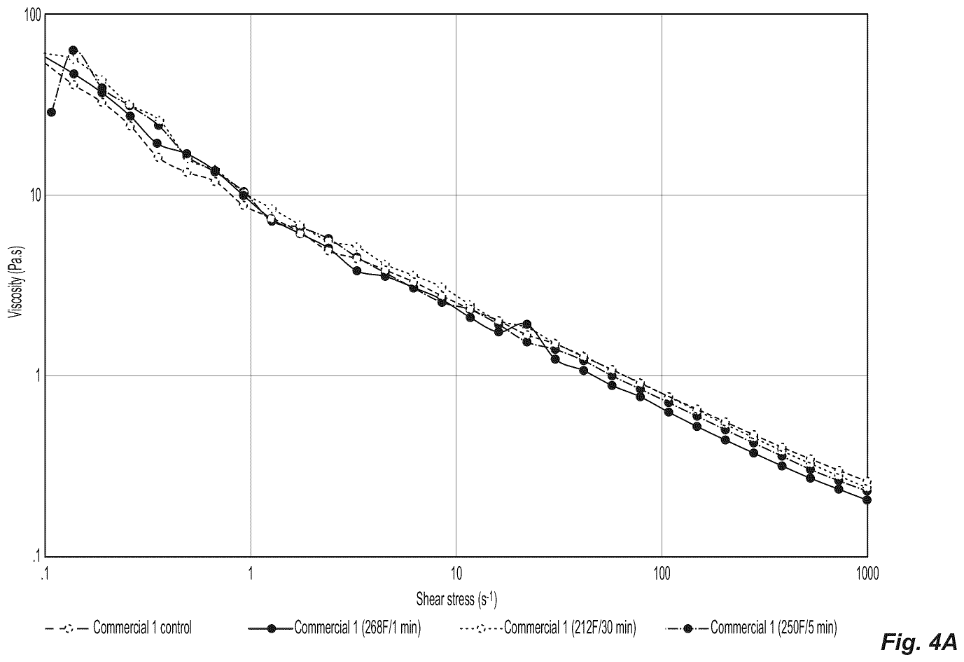

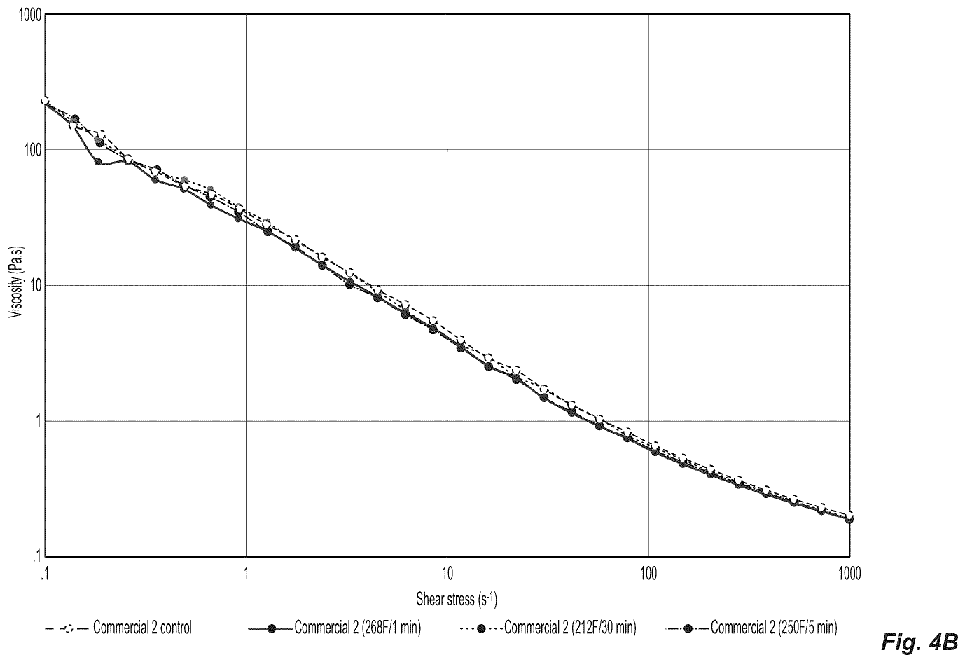

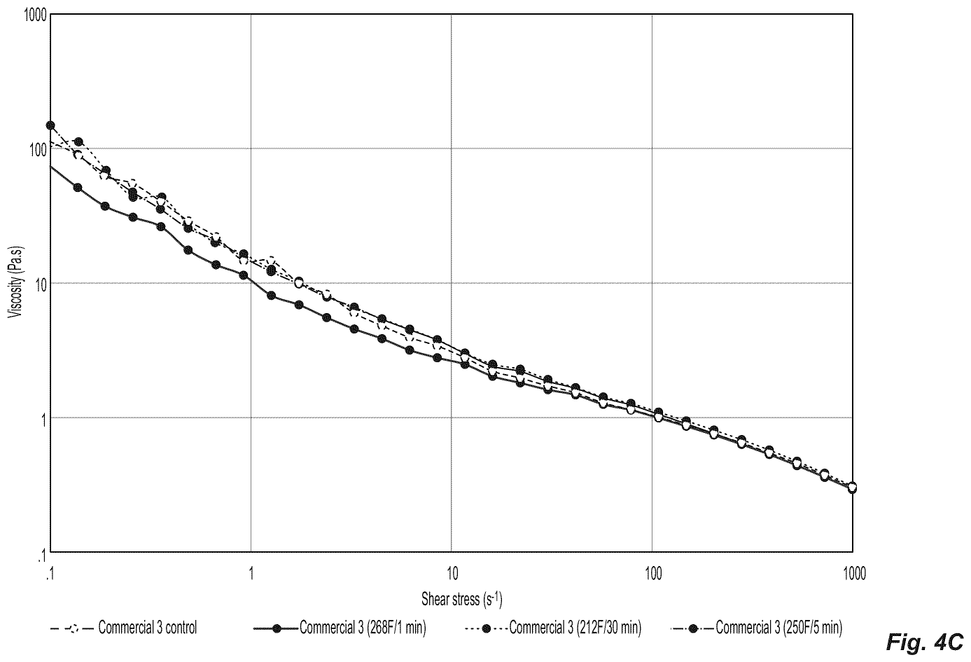

FIGS. 4A-C show that the flow curves for all three paint samples are substantially the same between the treated samples and the untreated control samples. The results show that paints and stains can be thermally treated at temperatures around and above 100° C. without loss of physical properties, e.g., rheological properties, and performance. Commercial paint 3 at 121° C. has a change of ICI viscosity near the upper range of 10% and still functions as shown in FIG. 4C.

The drawdown samples also show that treated commercial paints can satisfactorily form solid paint films on substrates, which is the primary function of paints and stains. The formation of trace amounts of skin/solid, observed in Experiments 1 and 2, does not negatively affect the treated paints' ability to cover substrates. Much higher amounts of skin are typically formed on open trays that hold paints, brushes/rollers and on the paint cans after they are opened.

Skin within unopened paint cans may form in certain situations, e.g., on the top of the aqueous paint inside the cans or on the lids. At higher storage temperatures, the airspace within the paint cans warms up faster than the aqueous paint due to the lower heat capacitance of air. This creates a temperature gradient or difference and water is driven off or evaporated at or near the top of the aqueous paint or lid creating trace amounts of skin.

With respect to the TiO₂'s reaction with the lining, TDT cans, which are similar to cans used to store foods, are typically lined with a polymeric materials to prevent foods from oxidizing or otherwise reacting with the cans. Common lining materials include BPA-based epoxy coating (bisphenol A+epichlorohydrin→bisphenol A-diglycidyl ether epoxy resin), epoxy amines, oleoresin plant-based material, vinyl (vinyl chloride and vinyl acetate), phenolic resin (phenol+aldehydes), acrylic, polyester (isophthalic acid (IPA) and terephthalic acid (TPA)) etc. Commercial paint cans do not have such lining and TiO₂ is not known to react with paint cans. Hence, this issue is not expected to occur with commercial paint cans.

The data in Table 8, the flow curves from FIGS. 4A-4C, and the high applied temperatures, as well as the high internal temperatures, show that paints, stains, and other architectural compositions can be heated at elevated temperatures of 100° C. (212° F.), 121° C. (250° F.) or 131° C. (268° F.) and higher for short time durations and retains their colloidal stability and rheological properties such as viscosity. These findings are unexpected and are contrary to the teachings of Rinno. The physical and performance properties of the latex binder in the paints, stains and other architectural compositions is preserved.

Experiment 2 shows that commercial paints 1, 2 and 3 can be heated to temperatures as high as 131° C. without losing their physical and performance properties. Hence, architectural coatings such as paints and stains can be flash pasteurized or flash sterilized to the HTST, HHST or UP regimes.

### Batch Processing and Continuous Processing

Paints and stains and other architectural compositions can be pasteurized in a batch process with the paints and stains stored inside containers being heat treated in batches, as described in Experiments 1 and 2. In another preferred embodiment, architectural compositions can be heat treated in a continuous fashion, e.g., in-line heating, followed by aseptic filling in soft or rigid, flexible, or metal containers that are sold to consumers.

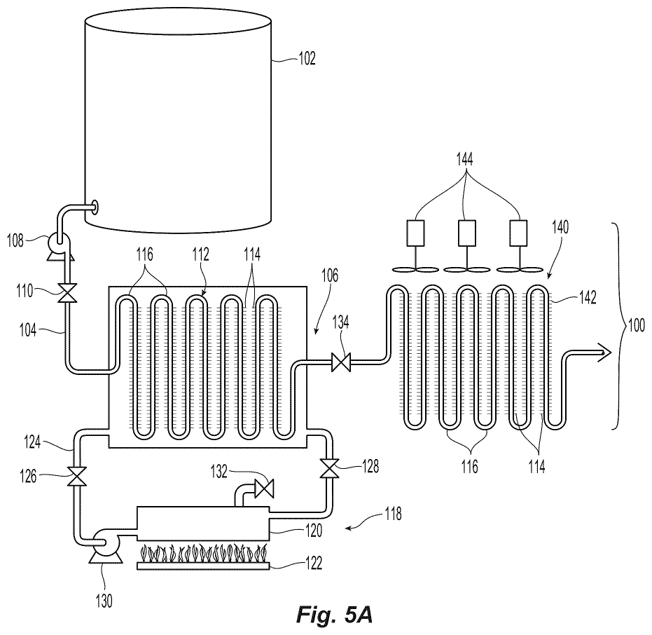

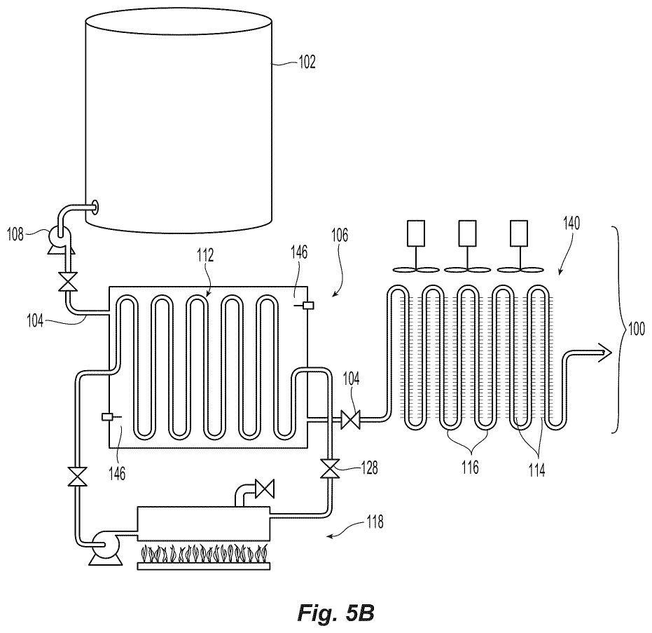

The architectural compositions can flow by gravity or pump pressure through the inside of piping, preferably serpentine piping, where heat is applied to the outside of the piping. Applied heat can be dry air heat, steam or heated water or other liquids. Preferably, heating fins with high heat transfer coefficient are attached to the outside of the piping to promote heat transfer between the heated air/liquid and the architectural compositions in the piping. Referring to FIGS. 5A and 5B, a continuous sterilization system is illustrated. Sterilization system 100 is connected to a tank 102 containing paints, stains or other architectural compositions to be pasteurized. A pipe 104 preferably connected proximate to a bottom of tank 102 connects tank 102 to a sterilization chamber 106. Preferably a pump 108 and valve 110 are installed on pipe 104 to transport the composition to be sterilized and to shut off the flow, respectively. First heat exchanger 112 is positioned within sterilization chamber 106. In the embodiment of FIG. 5A, pipe 104 is fluidly connected to first heat exchanger 112, so that the compositions to be sterilized flow inside the heat exchanger. Optionally, fins 114 can be provided on the outside of serpentine tube 116 to improve the heat transfer. A heating fluid circulates within sterilization chamber 106 outside of first heat exchanger 112 to heat the compositions flowing inside first heat exchanger 112. Sterilization chamber 106 preferably also has a heater 118 to heat the heating fluid to replace the heat transferred the compositions to sterilize same. Heater 118 may have a chamber 120 to receive the heating fluid. Chamber 120 may be heated by burner 122, which is preferably an electrical burner/wrapped blanket, or a combustion burner. Preferably sterilization chamber 106 is fluidly connected to heater 118 by pipe 124, with one or more valves 126 and 128 to control the flow of the heating fluid. The heating fluid can be a pressurized steam to provide heating temperature above 100° C., or water or other liquid to provide heating temperature up to 100° C. If water of another heating liquid is used, an optional pump 130 is provided and an optional refilling valve and inlet 132 are provided to add water or another heating liquid to chamber 120.

The volumetric flow rate and the time duration that the compositions to be sterilized spend within sterilization chamber 106 are calculated for said composition to reach and stay at or above the target pasteurization temperature, as taught above. After the sterilized compositions exit sterilization chamber 106, the compositions may optionally enter cooling zone 140, which includes a second heat exchanger 142, which is similar to first heat exchanger 112. Fans 144 are provided to force air through second heat exchanger 142 to cool via heat convection. Alternatively, pumps can provide cooling water. A valve 134 is preferably provided between sterilization chamber 106 and cooling zone 140. Cooling zone 140 may be omitted if sterilization by the aseptic technique, similar to that in Example 2, is selected.

An alternative embodiment is illustrated in FIG. 5B. This embodiment is similar to that shown in FIG. 5B, except that the compositions to be sterilized flows into sterilization chamber 106 outside of first heat exchanger 112. The heating fluid flows through heat exchanger 112 to heat the compositions to be sterilized. One or more mixers 146 is provided within sterilization chamber 146 to dynamically mix the compositions to enhance the heat transfer.

Suitable heat exchangers are disclosed in U.S. Pat. Nos. 9,395,121; 10,126,014; 9,568,212, etc. In certain embodiments, the heat exchangers may be similar to radiators for automobiles and trucks. The piping would be sized and dimensioned to adequately transport paints, stains and other architectural paints at desired volumetric rates. Internal flow weirs or vanes could be placed within the piping to promote flow circulations. Additionally, internal turbulence inducers, e.g., knubs or protrusions can be placed on the internal walls of the piping to promote turbulence flow, which provides more mixing than laminar flow.

The pasteurization process used in Example 1 has similarities to a continuous processing system in that cans of paint/stain are moved rotatably and translationally through the system similar to that of paint/stain being pumped through pipes.

One advantage of a continuous pasteurizing or sterilizing system, depending on the pasteurization temperature, is that it can affect a pasteurization process, as discussed in Experiment 1, or the HFH process in Experiment 2. Additionally, an aseptic process as discussed in Example 2 is possible, with higher heat and less exposure time without the holding step after the thermal step. Another advantage is that there should be no head space or air space in the piping with flowing paints and stains, which should minimize the formation of skin. Yet another advantage is that if any skin or solids are formed during the sterilization process, those could be filtered before the architectural compositions are placed in paint cans or buckets for sales.

The results from Experiments 1 and 2 can guide the continuous sterilization and/or pasteurization processes illustrated in connection with embodiments, such as those of FIGS. 5A and 5B. Experiment 1 shows that dynamic elevated heating of up to 100° C. applied temperature and up to 91° C. internal pasteurization temperature and mixing the paints or stains during the treatment do not significantly affect the colloidal stability and other properties of the treated paints and stains, as shown by the minor changes in viscosity after the DEH treatment. Experiment 2 pushes the knowledge envelope further by showing that heating the paints, stains and other architectural compositions to temperatures higher than about 100° C., higher than about 121° C. or about 131° C. in a short time period, such as less than 5 minutes or less than 1 minute, does not negatively affect the colloidal stability or other properties of the treated paints and stains, as shown by the minor changes in viscosity after the treatment.

This means that the continuous sterilization and/or pasteurization DEH processes can be designed such that the paints, stains and other architectural compositions being treated could be treated to internal temperatures higher than about 100° C., about 121° C. or about 131° C. in a short time, such as less than 5 minutes to less than 1 minute to optimize the sterilization and/or pasteurization processes. The volumetric flow rate of the architectural compositions and the level/amount of heat flux applied to the compositions being treated can be calculated according to known heat transfer principles to reach the desired time duration of the DEH treatment of the compositions.

### Experiment 3. Pasteurization/Sterilization Using a Continuous Process

Commercial #3 paint was successfully sterilized by heat at 132° C. (270° F.) for a bulk mean residence time of 15 seconds. Commercial #3 was also successfully sterilized by heat at 140° C. (285° F.) for a bulk mean residence time of 15 seconds, twice. The paints were pre-heated to 85° C. (185° F.) prior to being heated to the sterilization temperature. The paints were cooled afterward, and the treated samples were collected. (Heating temperatures of 131° C. in Experiment 2 and 132° C. in Experiment 3 are considered to be substantially the same temperature.)

Drawdown samples of the treated commercial #3 at 132° C. and 140° C. and untreated commercial #3 were compared and judged to form films in a similar fashion. The viscosity and pH of the treated paints are compared with those of the untreated paints and shown below. Multiple viscosity values of the treated paints and untreated paints were measured and averaged in the table below.

TABLE 9
Commercial #3

| | Stormer (KU) | ICI (P) | pH | Comments |
|---|---|---|---|---|
| untreated | 95.2 | 0.809 | 8.9 | |
| 15 sec @ | 94.6 | 0.788 | 8.9 | |
| 132° C. | (0.6%) | (2.6%) | | |
| 15 sec @ | 93.0 | 0.767 | 8.9 | Some skin formed |
| 140° C. | (2.3%) | (5.2%) | | in 1 of 4 cans |

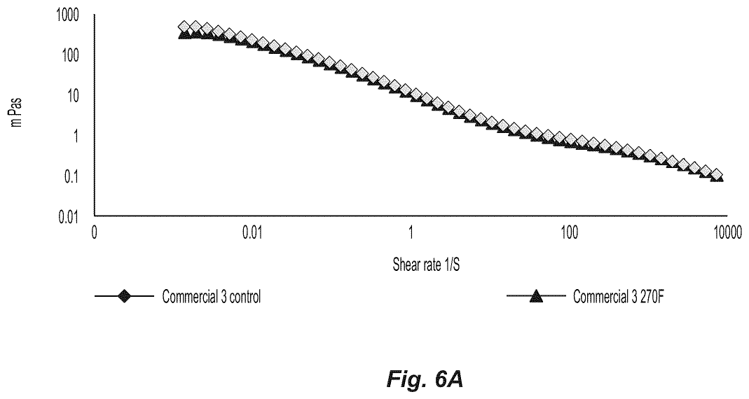

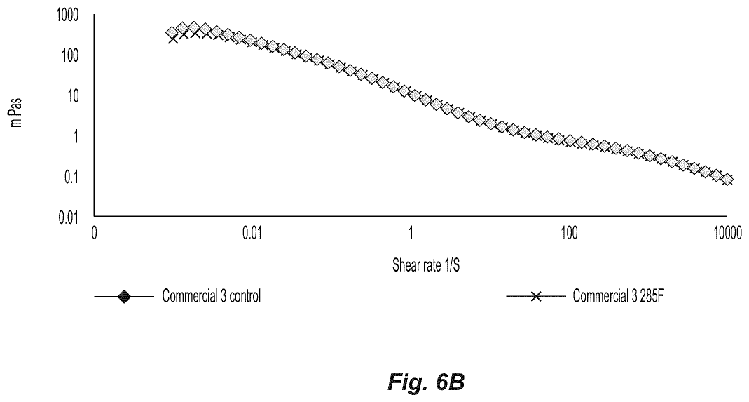

The flow curves shown in FIGS. 6A and 6B of the treated and untreated commercial #3 in Experiment 3 show no significant changes in the dynamic viscosity. The change in either the Stormer or ICI viscosity is within about 10% range, preferably within 7.5% range, preferably within 5% range and more preferably within 2.5% range, discussed above. It is noted that the 2.6% and 2.3% change in viscosity is within about 2.5% change and the 5.2% change is within about 5% change.

Commercial #2 paint was also tested at 132° C. Commercial #2 was pumped through the 85° C. preheater; however, the experimental equipment used in Experiment 3 was unable to process commercial #2 paint due to clogging at the principal heater. Experiment 2, discussed above, shows that commercial paints #2 was heated successfully to 131° C. for a longer time duration without forming skin. Hence, the present inventor believes that with redesigned commercial equipment including pumps, type of pumps (e.g., centrifugal vs piston), diameter of tubing/pipes and number and shape of bends in the tubing, etc., commercial paints #1 and #2 with higher pigment contents than commercial paint #3 can be pasteurized and/or sterilized at the temperatures used in Experiment 3.

### Storage of Paints, Stains and Other Architectural Compositions

Preferably, the storage of paint in step (iii) includes storing the paint containers in an environment in which bacteria, if present, do not grow. As discussed above, at 10° C. a large number of bacteria do not grow. Additionally, the temperature growth range of these bacteria is above 15° C., and very few bacteria would grow at a temperature of 20° C. Hence, preferably the paints are stored at temperature of 20° C. (68° F.) or lower, more preferably at temperature of 15° C. (59° F.) or lower, or more preferably at temperature of 10° C. (50° F.) or lower. These preferred storage temperatures can be achieved with conventional air conditioning technology. Additionally, it is preferred that during transportation the paints are also kept at these temperature ranges.

While it is apparent that the illustrative embodiments of the invention disclosed herein fulfill the objectives stated above, it is appreciated that numerous modifications and other embodiments may be devised by those skilled in the art. Therefore, it will be understood that the appended claims are intended to cover all such modifications and embodiments, which would come within the spirit and scope of the present invention.

APPENDIX
(All temperatures are in Fahrenheit—° F.)

| Time | Comm1 | Comm2 | Comm3 | TC1 | TC2 | TC3 | TC4 |
|---|---|---|---|---|---|---|---|
| 0:00:00 | 88.86 | 88.18 | 87.6 | 178.58 | 202.44 | 196.7 | 194.1 |
| 0:00:15 | 89.62 | 88.9 | 93.12 | 239.5 | 218.56 | 219.38 | 216.28 |
| 0:00:30 | 89.1 | 87.58 | 89.04 | 232.36 | 214.44 | 218.6 | 215.56 |
| 0:00:45 | 87.46 | 87.2 | 87.58 | 225.58 | 216.16 | 218.4 | 216.54 |
| 0:01:00 | 87.2 | 86.92 | 86.76 | 226 | 217.22 | 219.52 | 218.12 |
| 0:01:15 | 86.68 | 86.64 | 86.38 | 224.06 | 218.98 | 220.66 | 219.7 |
| 0:01:30 | 86.48 | 86.52 | 86.2 | 223.04 | 220.26 | 221.82 | 221 |
| 0:01:45 | 95.12 | 127.74 | 93.24 | 217.14 | 217.32 | 217.44 | 217.08 |
| 0:02:00 | 97.44 | 120.8 | 92.34 | 215.06 | 214.7 | 214.82 | 214.48 |
| 0:02:15 | 98.36 | 116.02 | 91.9 | 213.26 | 213.02 | 213.1 | 212.54 |
| 0:02:30 | 98.38 | 112.74 | 91.84 | 211.66 | 211.4 | 211.38 | 211.2 |
| 0:02:45 | 98.14 | 110.48 | 92 | 210.62 | 210.34 | 210.46 | 210.46 |
| 0:03:00 | 97.82 | 108.64 | 91.94 | 211.22 | 210.88 | 209.42 | 209.26 |
| 0:03:15 | 97.7 | 106.74 | 92.26 | 215.36 | 214.74 | 215.36 | 215.1 |
| 0:03:30 | 97.04 | 104.18 | 88.86 | 219.4 | 220.06 | 218.82 | 219.36 |
| 0:03:45 | 95.78 | 88.62 | 88 | 222.42 | 221.9 | 222.26 | 221.8 |
| 0:04:00 | 93.18 | 90.18 | 88.4 | 223.6 | 224.02 | 223.44 | 223.26 |
| 0:04:15 | 95.5 | 99.68 | 91.56 | 225.24 | 224.86 | 225.14 | 224.68 |
| 0:04:30 | 97.02 | 94.88 | 90.94 | 219.2 | 218.58 | 217.8 | 218.48 |
| 0:04:45 | 94.22 | 89.68 | 91.76 | 212.7 | 212.44 | 212.36 | 213.52 |
| 0:05:00 | 94.5 | 91.38 | 89.02 | 211.42 | 210.36 | 210.92 | 210.6 |
| 0:05:15 | 95.26 | 98.96 | 89.66 | 210.58 | 210.46 | 210.2 | 210.5 |
| 0:05:30 | 95.78 | 97.28 | 90.8 | 210.54 | 210.28 | 210.5 | 210.44 |
| 0:05:45 | 94.46 | 93.14 | 90.1 | 210.66 | 210.6 | 210.14 | 210.56 |
| 0:06:00 | 93.9 | 94.5 | 89.92 | 211 | 210.84 | 211.22 | 211.14 |
| 0:06:15 | 94.94 | 93.34 | 90.66 | 211.02 | 211.06 | 210.4 | 211.02 |
| 0:06:30 | 94.38 | 92.4 | 90.48 | 210.84 | 210.44 | 210.84 | 210.64 |
| 0:06:45 | 94.88 | 95.14 | 90.96 | 212.44 | 212.4 | 211.76 | 212.18 |
| 0:07:00 | 93.88 | 94.96 | 90.3 | 211.66 | 211.42 | 212.4 | 212.04 |
| 0:07:15 | 95.08 | 91.68 | 90.16 | 211.92 | 212.1 | 211.52 | 211.48 |
| 0:07:30 | 94.6 | 91.62 | 90 | 211.06 | 210.88 | 211.6 | 211.72 |
| 0:07:45 | 94.7 | 94.8 | 90.14 | 211.08 | 211 | 210.44 | 210.66 |
| 0:08:00 | 95.26 | 92.7 | 90.36 | 210.12 | 209.58 | 210.68 | 210.88 |
| 0:08:15 | 94.5 | 92.34 | 90.2 | 210.82 | 210.84 | 210.44 | 210 |
| 0:08:30 | 94.48 | 94.7 | 90.56 | 209.7 | 210.1 | 210.46 | 210.84 |
| 0:08:45 | 94.94 | 93.24 | 90.98 | 210.6 | 210.3 | 210.18 | 209.94 |
| 0:09:00 | 94.48 | 93.78 | 90.8 | 210.02 | 210.66 | 210.34 | 210.78 |
| 0:09:15 | 94.84 | 92.54 | 90.98 | 210.94 | 210.24 | 210.58 | 210 |
| 0:09:30 | 94.72 | 91.82 | 91.86 | 209.76 | 210.66 | 210.28 | 211.06 |
| 0:09:45 | 95 | 92.72 | 90.98 | 210.74 | 210.14 | 210.8 | 209.92 |
| 0:10:00 | 95.26 | 94.58 | 91.34 | 210.08 | 210.94 | 210.14 | 211.08 |
| 0:10:15 | 95.38 | 93.98 | 91.36 | 211 | 210.02 | 210.98 | 210.08 |
| 0:10:30 | 94.94 | 91.78 | 91.24 | 210.14 | 211.04 | 211.06 | 211.12 |
| 0:10:45 | 95.18 | 92.16 | 91.1 | 210.62 | 210.38 | 210.8 | 210.74 |
| 0:11:00 | 95.66 | 93.72 | 91.58 | 210.66 | 210.98 | 209.96 | 210.5 |
| 0:11:15 | 95.16 | 93.38 | 91.28 | 210.4 | 209.96 | 211.06 | 210.8 |
| 0:11:30 | 95.4 | 92.42 | 91.5 | 210.82 | 210.86 | 209.92 | 210.48 |
| 0:11:45 | 96.12 | 93 | 91.22 | 210.28 | 209.94 | 211.06 | 210.86 |
| 0:12:00 | 96.54 | 93.02 | 91.52 | 210.94 | 210.92 | 210.14 | 210.12 |
| 0:12:15 | 95.36 | 93.14 | 92.52 | 210.06 | 210.16 | 211.2 | 211.04 |
| 0:12:30 | 97.16 | 93.42 | 91.5 | 210.98 | 211.3 | 210.18 | 210.78 |
| 0:12:45 | 96.36 | 93.34 | 91.58 | 209.92 | 210.84 | 210.6 | 211.22 |
| 0:13:00 | 95.78 | 93.7 | 92.32 | 211 | 210.94 | 210.94 | 210.26 |
| 0:13:15 | 96.2 | 93.76 | 92.74 | 209.88 | 210.94 | 210.68 | 211.34 |
| 0:13:30 | 96.1 | 93.76 | 92.38 | 211.04 | 210.38 | 211.04 | 209.9 |
| 0:13:45 | 98.36 | 94.04 | 93.22 | 209.74 | 211.18 | 210.92 | 211.3 |
| 0:14:00 | 96.96 | 93.6 | 92.88 | 211.14 | 210.58 | 211.16 | 210.5 |
| 0:14:15 | 99.48 | 94.62 | 93.66 | 210.18 | 211.12 | 210.28 | 211.32 |
| 0:14:30 | 102.54 | 94.02 | 93.02 | 211.4 | 210.42 | 211.24 | 210.6 |
| 0:14:45 | 109.82 | 95.78 | 94.86 | 210.76 | 211.22 | 210.62 | 211.4 |
| 0:15:00 | 100.74 | 95.5 | 93.34 | 210.68 | 210.4 | 211.22 | 210.92 |
| 0:15:15 | 97.22 | 95.52 | 95.72 | 210.64 | 210.84 | 210.52 | 210.74 |
| 0:15:30 | 104.44 | 95.46 | 93.58 | 210.48 | 210.3 | 211.2 | 210.88 |
| 0:15:45 | 101.96 | 96.58 | 93.98 | 211.02 | 211.32 | 210.42 | 210.68 |
| 0:16:00 | 110.12 | 95.02 | 96.14 | 210.74 | 210.42 | 211.28 | 211.24 |
| 0:16:15 | 107.56 | 96.78 | 93.98 | 211.04 | 211.36 | 210.3 | 210.72 |
| 0:16:30 | 103.78 | 96.78 | 94.38 | 210.3 | 210.42 | 211.22 | 211.1 |
| 0:16:45 | 113.4 | 98.82 | 94.3 | 211.02 | 210.92 | 210.2 | 210.78 |
| 0:17:00 | 106.12 | 96.62 | 97.62 | 210.06 | 210.7 | 210.36 | 210.88 |
| 0:17:15 | 113 | 97.76 | 96.38 | 210.72 | 210.6 | 210.68 | 210.08 |
| 0:17:30 | 107.22 | 98.46 | 96.58 | 209.9 | 210.94 | 210.52 | 210.96 |
| 0:17:45 | 113.86 | 99.96 | 96.64 | 210.42 | 210.12 | 209.78 | 209.88 |
| 0:18:00 | 111.92 | 98.76 | 98.18 | 209.96 | 210.98 | 210.34 | 210.8 |
| 0:18:15 | 110.56 | 98.34 | 98 | 210.64 | 210 | 210.84 | 210.24 |
| 0:18:30 | 112.84 | 99.28 | 106 | 209.62 | 210.72 | 209.68 | 210.7 |
| 0:18:45 | 111.84 | 99.46 | 98.9 | 210.46 | 210.22 | 210.66 | 209.82 |
| 0:19:00 | 115.8 | 100.88 | 101.04 | 209.76 | 210.58 | 209.74 | 210.74 |
| 0:19:15 | 108.2 | 100.3 | 99.44 | 209.98 | 209.78 | 210.6 | 210.2 |
| 0:19:30 | 121.66 | 102.22 | 106.1 | 210.18 | 210.3 | 209.7 | 210.38 |
| 0:19:45 | 108.72 | 101.02 | 99.78 | 209.98 | 209.62 | 210.52 | 210.4 |
| 0:20:00 | 124.96 | 102.28 | 106.96 | 210.56 | 210.92 | 210.04 | 210.24 |
| 0:20:15 | 113.92 | 100.9 | 99.6 | 209.94 | 210.04 | 210.82 | 210.78 |
| 0:20:30 | 125.04 | 103.5 | 101.72 | 210.68 | 210.92 | 210.1 | 210.34 |
| 0:20:45 | 116.26 | 101.44 | 102.18 | 210.16 | 210.08 | 211.16 | 210.98 |
| 0:21:00 | 127.12 | 104.32 | 106.22 | 210.94 | 211.26 | 210.38 | 210.4 |
| 0:21:15 | 110.76 | 102.52 | 108.44 | 211.42 | 210.16 | 210.68 | 210.82 |
| 0:21:30 | 123.92 | 104.9 | 105.78 | 210.7 | 210.7 | 210.7 | 209.7 |
| 0:21:45 | 111.6 | 102.92 | 107.96 | 210.16 | 210.92 | 210.56 | 210.92 |
| 0:22:00 | 122 | 105.96 | 105.96 | 211.02 | 210.64 | 211.04 | 210.18 |
| 0:22:15 | 120.16 | 105.72 | 109.2 | 210.12 | 210.86 | 210.36 | 211.34 |
| 0:22:30 | 123.08 | 107.02 | 105.9 | 210.9 | 210.46 | 210.88 | 210.42 |
| 0:22:45 | 124.92 | 105.08 | 112.46 | 210.26 | 210.98 | 210.56 | 211.24 |
| 0:23:00 | 122.26 | 107.24 | 109.68 | 210.74 | 210.12 | 211.1 | 210.74 |
| 0:23:15 | 129.18 | 107.34 | 111.1 | 210.64 | 210.96 | 209.98 | 211.26 |
| 0:23:30 | 122.16 | 107.6 | 116.32 | 211.12 | 210.44 | 210.92 | 210.4 |
| 0:23:45 | 132.96 | 108.98 | 132.56 | 210.26 | 211 | 210.48 | 211.18 |
| 0:24:00 | 117.08 | 108.16 | 110.32 | 210.62 | 210.5 | 211.3 | 211.04 |
| 0:24:15 | 134.3 | 109.52 | 115.04 | 210.96 | 211.34 | 210.46 | 210.78 |
| 0:24:30 | 123.34 | 108.14 | 118.88 | 210.76 | 210.48 | 211.18 | 211.08 |
| 0:24:45 | 140.92 | 112.46 | 123.54 | 211.04 | 211.04 | 210.6 | 210.74 |
| 0:25:00 | 122.66 | 107.64 | 114.58 | 210.86 | 210.82 | 211.46 | 211.3 |
| 0:25:15 | 135.92 | 111.18 | 131.26 | 211.36 | 211.6 | 211.08 | 211.14 |
| 0:25:30 | 125.38 | 108.9 | 115.38 | 211.06 | 210.8 | 211.48 | 211.4 |
| 0:25:45 | 134.34 | 112.12 | 118.26 | 211.22 | 211.42 | 210.7 | 210.64 |
| 0:26:00 | 121.8 | 109.8 | 121.9 | 210.82 | 211.32 | 211.26 | 211.4 |
| 0:26:15 | 133.96 | 113.34 | 130.92 | 211.26 | 211.5 | 210.78 | 210.94 |
| 0:26:30 | 122.7 | 111.06 | 125.58 | 211.06 | 211.24 | 211.2 | 211.28 |
| 0:26:45 | 129.88 | 113.12 | 132.3 | 211.32 | 210.96 | 211.42 | 210.76 |
| 0:27:00 | 126.9 | 112.38 | 121.14 | 210.5 | 211.32 | 210.84 | 211.52 |
| 0:27:15 | 130.88 | 115.3 | 129.16 | 211.24 | 210.86 | 211.32 | 211.2 |
| 0:27:30 | 131.44 | 113.04 | 130.96 | 210.62 | 211.3 | 210.58 | 211.58 |
| 0:27:45 | 130.6 | 114.12 | 131.6 | 210.94 | 210.56 | 211.38 | 210.68 |
| 0:28:00 | 135.66 | 116.26 | 141.52 | 210.76 | 211.44 | 211.1 | 211.56 |
| 0:28:15 | 131.08 | 115.38 | 132.68 | 211.52 | 210.84 | 211.28 | 210.8 |
| 0:28:30 | 137.4 | 115.16 | 125.98 | 210.68 | 211.28 | 210.48 | 211.26 |
| 0:28:45 | 127.54 | 115.62 | 135.98 | 210.72 | 210.92 | 211.24 | 211.2 |
| 0:29:00 | 140.64 | 118.52 | 139.5 | 211.1 | 211.4 | 210.64 | 210.94 |
| 0:29:15 | 127.86 | 116.06 | 126.78 | 210.86 | 210.72 | 211.26 | 210.84 |
| 0:29:30 | 141.12 | 119.12 | 142.68 | 210.92 | 211.26 | 210.88 | 210.92 |
| 0:29:45 | 131.36 | 116.66 | 132.12 | 210.44 | 210.5 | 211.28 | 211.1 |
| 0:30:00 | 141.22 | 122.74 | 134.14 | 210.9 | 211.2 | 210.52 | 210.52 |
| 0:30:15 | 135.98 | 116.38 | 141.72 | 210.38 | 210.74 | 211.28 | 211.16 |
| 0:30:30 | 141.54 | 120.46 | 145 | 210.9 | 211.32 | 210.56 | 211.1 |
| 0:30:45 | 128.64 | 119.92 | 133.72 | 210.76 | 210.74 | 211.14 | 211.24 |
| 0:31:00 | 136.96 | 125.58 | 141.94 | 211.06 | 210.88 | 210.66 | 210.18 |
| 0:31:15 | 129.5 | 117.6 | 139.52 | 210.98 | 211.06 | 210.82 | 211.18 |
| 0:31:30 | 141.02 | 121.86 | 147.88 | 211.02 | 211.3 | 210.6 | 210.88 |
| 0:31:45 | 130.1 | 122.64 | 145.14 | 210.88 | 211.06 | 211.02 | 211.14 |
| 0:32:00 | 137.14 | 123.94 | 147.58 | 211.28 | 210.94 | 211.24 | 210.88 |
| 0:32:15 | 133.38 | 122.48 | 137.12 | 210.54 | 211.12 | 210.78 | 211.26 |
| 0:32:30 | 140.5 | 125.08 | 142.98 | 211.1 | 210.9 | 211.18 | 210.66 |
| 0:32:45 | 134.82 | 123.18 | 145.9 | 210.54 | 211.26 | 210.7 | 211.2 |
| 0:33:00 | 140.12 | 126.34 | 152.42 | 211.26 | 210.7 | 211.28 | 210.66 |
| 0:33:15 | 137.84 | 125.52 | 156.06 | 210.44 | 211.26 | 210.62 | 211.48 |
| 0:33:30 | 139.48 | 125.98 | 153 | 211.3 | 210.78 | 211.34 | 210.86 |
| 0:33:45 | 144.78 | 128.26 | 142.92 | 210.64 | 211.22 | 210.88 | 211.36 |
| 0:34:00 | 139 | 126.54 | 152.16 | 210.9 | 210.84 | 211.4 | 210.78 |
| 0:34:15 | 142.88 | 127.82 | 153.48 | 210.78 | 211.38 | 210.88 | 211.08 |
| 0:34:30 | 140.86 | 129.12 | 146.96 | 211 | 211.04 | 211.34 | 210.88 |
| 0:34:45 | 144.38 | 130.84 | 159.56 | 211.16 | 211.36 | 210.8 | 210.9 |
| 0:35:00 | 141.44 | 126.72 | 147.64 | 210.68 | 210.54 | 211.44 | 211.36 |
| 0:35:15 | 147.82 | 131.76 | 148.32 | 211.24 | 211.4 | 210.76 | 211.02 |
| 0:35:30 | 137 | 127.82 | 154.52 | 210.66 | 210.72 | 211.46 | 211.4 |
| 0:35:45 | 146.7 | 132.6 | 156.08 | 211.2 | 211.42 | 210.88 | 210.68 |
| 0:36:00 | 138.36 | 130.66 | 155.94 | 210.5 | 210.8 | 211.34 | 211.38 |
| 0:36:15 | 149 | 136.7 | 160.7 | 211.18 | 211.5 | 211.06 | 210.78 |
| 0:36:30 | 139.06 | 129.12 | 151.88 | 210.5 | 211.32 | 211.64 | 211.44 |
| 0:36:45 | 145.96 | 135 | 161.22 | 211.26 | 211.28 | 211.34 | 210.88 |
| 0:37:00 | 139.76 | 133.32 | 159.52 | 209.9 | 211.12 | 211.28 | 211.28 |
| 0:37:15 | 146.98 | 140.02 | 158.22 | 211.06 | 210.96 | 211.18 | 210.76 |
| 0:37:30 | 144.6 | 134.82 | 159.84 | 210.3 | 211.18 | 210.8 | 211.4 |
| 0:37:45 | 147.86 | 140.32 | 161.1 | 211.18 | 210.94 | 211.18 | 210.9 |
| 0:38:00 | 147.16 | 139.46 | 154.88 | 210.46 | 211.22 | 210.62 | 211.32 |
| 0:38:15 | 147.62 | 141.68 | 160.86 | 211.2 | 210.76 | 211.26 | 210.72 |
| 0:38:30 | 145.6 | 138.4 | 161.8 | 210.46 | 211.42 | 210.86 | 211.7 |
| 0:38:45 | 149.32 | 137.9 | 161.46 | 211.42 | 210.7 | 211.4 | 210.84 |
| 0:39:00 | 150.48 | 141.36 | 157.04 | 210.46 | 211.32 | 210.7 | 211.56 |
| 0:39:15 | 150.26 | 142.06 | 162.36 | 210.78 | 210.92 | 211.34 | 211.1 |
| 0:39:30 | 151.32 | 142.7 | 164.38 | 210.92 | 211.26 | 210.34 | 211.2 |
| 0:39:45 | 147.68 | 140.94 | 159.84 | 210.84 | 211.18 | 211.3 | 211.16 |
| 0:40:00 | 158.22 | 145.7 | 167.62 | 210.98 | 211.22 | 210.86 | 211.1 |
| 0:40:15 | 146.4 | 142.6 | 160.52 | 210.72 | 210.46 | 211.2 | 211.04 |
| 0:40:30 | 159.36 | 146.84 | 159.94 | 211.06 | 211.14 | 210.56 | 210.94 |
| 0:40:45 | 148.58 | 144.14 | 166.46 | 210.66 | 210.78 | 211.22 | 211.14 |
| 0:41:00 | 157.46 | 150.12 | 165.86 | 210.98 | 211.5 | 210.72 | 210.7 |
| 0:41:15 | 155.24 | 145.18 | 163.24 | 210.46 | 210.58 | 211.26 | 211.14 |
| 0:41:30 | 159.56 | 152.68 | 168.04 | 211.06 | 211.26 | 210.94 | 210.56 |
| 0:41:45 | 152.06 | 147.96 | 162.68 | 210.5 | 211.06 | 211.4 | 211.32 |
| 0:42:00 | 157.94 | 155.32 | 168.96 | 211.22 | 210.9 | 211.1 | 210.94 |
| 0:42:15 | 149.38 | 148.84 | 167.44 | 210.76 | 211.18 | 211 | 211.3 |
| 0:42:30 | 160.56 | 154.9 | 168.52 | 211.12 | 211.02 | 211.28 | 210.72 |
| 0:42:45 | 149.48 | 153.6 | 167.7 | 210.12 | 211 | 211.08 | 211.3 |
| 0:43:00 | 162.14 | 158.18 | 165.6 | 211.32 | 211.06 | 211.42 | 210.96 |
| 0:43:15 | 153.14 | 153.04 | 164.48 | 210.82 | 211.36 | 211.1 | 211.54 |
| 0:43:30 | 163.68 | 159.52 | 165.94 | 211.46 | 210.96 | 211.52 | 210.96 |
| 0:43:45 | 161.18 | 158.98 | 168.12 | 210.94 | 211.48 | 210.94 | 211.86 |
| 0:44:00 | 164.3 | 162.3 | 168.68 | 211.36 | 211.18 | 211.44 | 210.94 |
| 0:44:15 | 161.12 | 162.8 | 169.08 | 210.76 | 211.42 | 210.98 | 211.74 |
| 0:44:30 | 166.06 | 164.28 | 171.04 | 211.44 | 211 | 211.4 | 211.2 |
| 0:44:45 | 170.52 | 165.02 | 166.14 | 211.14 | 211.34 | 210.94 | 211.16 |
| 0:45:00 | 162.22 | 163.7 | 171.06 | 211.2 | 211.54 | 211.36 | 211.14 |
| 0:45:15 | 165.02 | 167.36 | 170.78 | 211.2 | 211.42 | 210.66 | 211.5 |
| 0:45:30 | 167.98 | 167.34 | 167.68 | 211.18 | 210.88 | 211.36 | 211.3 |
| 0:45:45 | 168.66 | 169.54 | 170.66 | 211.28 | 211.4 | 210.52 | 211.34 |
| 0:46:00 | 165.98 | 165.64 | 168.86 | 210.82 | 210.92 | 211.38 | 211.34 |
| 0:46:15 | 174.4 | 172.74 | 168.86 | 211.28 | 211.36 | 211.08 | 211.16 |
| 0:46:30 | 163.94 | 168.6 | 172.36 | 211.04 | 211 | 211.44 | 211.4 |
| 0:46:45 | 175.8 | 175.24 | 172.8 | 211.26 | 211.52 | 211 | 210.98 |
| 0:47:00 | 170.7 | 170.48 | 168.5 | 210.82 | 210.84 | 211.48 | 211.34 |
| 0:47:15 | 175.78 | 177.64 | 170.82 | 211.14 | 211.18 | 211.04 | 211.2 |
| 0:47:30 | 167.5 | 173.38 | 170.98 | 210.68 | 210.76 | 211.5 | 211.32 |
| 0:47:45 | 171.34 | 179.28 | 170 | 204.58 | 203.32 | 205.26 | 205.08 |
| 0:48:00 | 170.42 | 179.82 | 170.56 | 201.7 | 199.42 | 203.76 | 202.4 |
| 0:48:15 | 169.34 | 179.38 | 170.02 | 198.74 | 191.74 | 199 | 198.02 |
| 0:48:30 | 168.6 | 179.02 | 170.06 | 193.76 | 183.6 | 194.18 | 191.98 |
| 0:48:45 | 167.96 | 179.3 | 170.1 | 189.58 | 177.26 | 189.56 | 183.94 |
| 0:49:00 | 167.74 | 179.82 | 169.86 | 186.48 | 171.04 | 186.08 | 178.96 |
| 0:49:15 | 167.18 | 179.54 | 170.38 | 182.48 | 167.5 | 180.88 | 173.66 |
| 0:49:30 | 167.04 | 179.7 | 170.84 | 179.72 | 160.54 | 177.08 | 169.3 |
| 0:49:45 | 166.94 | 179.6 | 170.62 | 177.48 | 157.5 | 171.8 | 164.5 |
| 0:50:00 | 166.68 | 179.68 | 170.6 | 175.18 | 152.98 | 168.36 | 160.64 |
| 0:50:15 | 167.22 | 179.72 | 170.82 | 171.7 | 146.92 | 163.76 | 157.14 |
| 0:50:30 | 166.76 | 179.68 | 170.76 | 168.68 | 143.02 | 160.78 | 153.16 |
| 0:50:45 | 166.42 | 180.14 | 171.1 | 165.78 | 139.66 | 156.94 | 146.12 |
| 0:51:00 | 166.42 | 180.3 | 170.92 | 163.7 | 137.18 | 154.7 | 145.58 |
| 0:51:15 | 167.22 | 180.52 | 171.02 | 160.46 | 133.72 | 151.36 | 140.58 |
| 0:51:30 | 167.48 | 180.64 | 170.84 | 159.58 | 133.4 | 151.64 | 138.76 |
| 0:51:45 | 167.3 | 180.82 | 171.12 | 158.04 | 132.08 | 148.88 | 138.22 |
| 0:52:00 | 167.72 | 181.02 | 171 | 158.52 | 129.82 | 149.06 | 130.14 |
| 0:52:15 | 167.46 | 181.36 | 171.2 | 156.9 | 128.66 | 146.64 | 129.82 |
| 0:52:30 | 167.04 | 181.54 | 170.98 | 155.96 | 127.8 | 146.1 | 129.42 |
| 0:52:45 | 167.18 | 181.82 | 170.96 | 156.42 | 126.46 | 145.4 | 128.54 |
| 0:53:00 | 166.96 | 182.08 | 171.22 | 155.7 | 125.7 | 147.32 | 127.04 |
| 0:53:15 | 167.04 | 182.18 | 171.12 | 154.4 | 124.54 | 148.08 | 126.46 |
| 0:53:30 | 167.42 | 182.48 | 171.26 | 152.82 | 122.88 | 148.2 | 124.96 |
| 0:53:45 | 172.56 | 181.44 | 171.36 | 121.68 | 122 | 148.4 | 128.76 |
| 0:54:00 | 170.6 | 181.16 | 171.54 | 130.1 | 130.02 | 118.54 | 120.6 |
| 0:54:15 | 172.12 | 181.28 | 172.16 | 120.26 | 118.62 | 128.84 | 127.02 |
| 0:54:30 | 171.78 | 181.94 | 171.44 | 123.76 | 128.3 | 115.72 | 123.96 |
| 0:54:45 | 172.46 | 181.98 | 172.14 | 118.88 | 114.04 | 124.92 | 122.42 |
| 0:55:00 | 171.74 | 181.74 | 171.88 | 122.54 | 125.14 | 113.68 | 118.3 |
| 0:55:15 | 169.82 | 183.66 | 172.02 | 117.34 | 114.12 | 121.74 | 121.22 |
| 0:55:30 | 169.86 | 181.62 | 172.02 | 119.54 | 123.36 | 112.08 | 119.26 |
| 0:55:45 | 171.36 | 185.62 | 172.3 | 115.78 | 111.12 | 120.44 | 118.1 |
| 0:56:00 | 171.76 | 182.88 | 171.86 | 120.78 | 118.78 | 111.08 | 110.42 |
| 0:56:15 | 170.16 | 187.58 | 172.26 | 118.16 | 108.88 | 119.32 | 113.64 |
| 0:56:30 | 170.32 | 185.86 | 171.58 | 118.38 | 117.54 | 108.98 | 109.12 |
| 0:56:45 | 170.12 | 190 | 172 | 113.7 | 106.76 | 117.42 | 113.28 |
| 0:57:00 | 171.46 | 188.7 | 172.42 | 118.14 | 115.2 | 107.46 | 106.8 |
| 0:57:15 | 170.7 | 191.62 | 172.3 | 117.24 | 105.36 | 116.64 | 111.18 |
| 0:57:30 | 170.54 | 192.56 | 172.2 | 117.06 | 114.46 | 105.42 | 106.04 |
| 0:57:45 | 170.42 | 193.52 | 172.44 | 110.36 | 103.66 | 114.18 | 110.58 |
| 0:58:00 | 171 | 193.86 | 172.16 | 116 | 112.64 | 104.68 | 103.76 |
| 0:58:15 | 170.9 | 194.44 | 172.36 | 108.34 | 102.34 | 112.4 | 109.88 |
| 0:58:30 | 171.28 | 195.7 | 172.44 | 111.96 | 112.6 | 102.26 | 102.6 |
| 0:58:45 | 170.84 | 195.78 | 172.36 | 106.82 | 102.3 | 111.18 | 111.76 |
| 0:59:00 | 170.96 | 195.62 | 172.68 | 115.78 | 110.3 | 102.9 | 100.58 |
| 0:59:15 | 171.56 | 195.78 | 172.44 | 105.84 | 99.72 | 111.1 | 106.9 |
| 0:59:30 | 171.78 | 193.7 | 172.68 | 113.44 | 109.06 | 100.92 | 99.16 |
| 0:59:45 | 171.56 | 194.18 | 172.5 | 105.3 | 97.98 | 110.5 | 104.68 |
| 1:00:00 | 171.36 | 190.1 | 172.76 | 113.52 | 107.08 | 99.36 | 98.3 |
| 1:00:15 | 171.18 | 192.82 | 172.56 | 103.32 | 97.56 | 109.16 | 104.8 |
| 1:00:30 | 171.92 | 188.76 | 172.88 | 109.52 | 107.68 | 98.42 | 97.26 |
| 1:00:45 | 171.44 | 190.54 | 172.58 | 102.02 | 98 | 107.1 | 105.52 |
| 1:01:00 | 172.12 | 183.66 | 172.68 | 110.24 | 106.12 | 97.6 | 96.22 |
| 1:01:15 | 172.66 | 189.04 | 172.68 | 100.86 | 98.34 | 105.16 | 105.74 |
| 1:01:30 | 170.6 | 181.12 | 172.36 | 110.18 | 104.54 | 98 | 95.14 |
| 1:01:45 | 172.64 | 185.8 | 172.74 | 99.8 | 97.38 | 103.68 | 106.16 |
| 1:02:00 | 174.94 | 177.94 | 172.8 | 109.24 | 104.38 | 96.38 | 94.3 |
| 1:02:15 | 172.44 | 183.1 | 172.84 | 99.32 | 97.6 | 102.88 | 103.94 |
| 1:02:30 | 169.86 | 176.38 | 173.04 | 109 | 102.94 | 96.48 | 93.28 |
| 1:02:45 | 174.78 | 180.3 | 172.92 | 97.84 | 96.1 | 102.34 | 105.68 |
| 1:03:00 | 172.68 | 173.78 | 172.64 | 107.28 | 102.78 | 95.2 | 92.76 |
| 1:03:15 | 165.48 | 178.38 | 173 | 97.04 | 96 | 100.86 | 103.44 |
| 1:03:30 | 161.16 | 172.44 | 173.1 | 107.44 | 100.96 | 95.56 | 91.72 |
| 1:03:45 | 144.04 | 175.48 | 173.04 | 96.48 | 92.9 | 102.24 | 100.16 |
| 1:04:00 | 141.18 | 170.08 | 173.18 | 106.4 | 100.2 | 94.12 | 91.06 |
| 1:04:15 | 134.84 | 173.54 | 173.16 | 95.62 | 94.08 | 100.26 | 102.52 |
| 1:04:30 | 136.72 | 170.8 | 173.72 | 102.8 | 101.9 | 92.4 | 90.74 |
| 1:04:45 | 130.04 | 172 | 173.28 | 95.08 | 93.32 | 100.58 | 100.78 |
| 1:05:00 | 128.12 | 168.02 | 172.96 | 105.68 | 98.28 | 94.38 | 89.96 |
| 1:05:15 | 135.4 | 170.14 | 173.34 | 94.36 | 93.16 | 98.98 | 101.52 |
| 1:05:30 | 141.22 | 166.44 | 173.38 | 105.4 | 98.24 | 93.3 | 89.36 |
| 1:05:45 | 125.46 | 168.66 | 173.56 | 93.98 | 93.28 | 98.18 | 100.24 |
| 1:06:00 | 125.9 | 165.68 | 173.46 | 104.1 | 98.22 | 92.5 | 89.04 |
| 1:06:15 | 122.48 | 167.12 | 173.6 | 92.92 | 92.26 | 97.16 | 100.18 |
| 1:06:30 | 122.92 | 164.1 | 173.5 | 103.32 | 97.92 | 91.92 | 88.48 |
| 1:06:45 | 122.34 | 165.56 | 173.74 | 92.44 | 92.34 | 96.46 | 98.18 |
| 1:07:00 | 123.04 | 163.22 | 173.68 | 101.98 | 97.06 | 91.78 | 88.1 |
| 1:07:15 | 120.4 | 164.08 | 173.88 | 91.68 | 91.62 | 96.1 | 98.3 |
| 1:07:30 | 119.54 | 162.54 | 173.8 | 99.22 | 97.6 | 90.3 | 87.72 |
| 1:07:45 | 119.38 | 162.88 | 173.98 | 91.04 | 91.02 | 95.18 | 98.64 |
| 1:08:00 | 120.86 | 160.82 | 173.88 | 101.32 | 95.62 | 91.28 | 87.26 |
| 1:08:15 | 119.52 | 161.7 | 174.28 | 90.42 | 90.62 | 94.78 | 98.16 |
| 1:08:30 | 120.72 | 160.02 | 174.68 | 100.4 | 95.84 | 90.4 | 86.92 |
| 1:08:45 | 118.12 | 160.52 | 174.66 | 89.72 | 91.1 | 94.92 | 98.22 |
| 1:09:00 | 117.46 | 158.48 | 174.58 | 99.56 | 94.6 | 90.64 | 86.56 |
| 1:09:15 | 117.66 | 159.46 | 174.98 | 89.18 | 90.48 | 93.88 | 97.18 |
| 1:09:30 | 119.24 | 157.72 | 174.92 | 99.02 | 94.3 | 89.7 | 86.22 |
| 1:09:45 | 116.74 | 158.48 | 175.18 | 88.48 | 90.46 | 93.02 | 96.18 |
| 1:10:00 | 116.68 | 156.32 | 175.2 | 98.32 | 93.46 | 90.38 | 85.98 |
| 1:10:15 | 115.96 | 157.4 | 174.84 | 88.42 | 89.08 | 93.76 | 96.06 |
| 1:10:30 | 116.9 | 156.02 | 175.12 | 98.74 | 93.22 | 89.7 | 85.58 |
| 1:10:45 | 115.24 | 156.46 | 174.28 | 87.68 | 89.28 | 92.44 | 96.52 |
| 1:11:00 | 116.24 | 155.42 | 172.56 | 97.94 | 92.52 | 89.84 | 85.3 |
| 1:11:15 | 114.48 | 155.76 | 173.4 | 87.32 | 88.92 | 93.08 | 96.1 |
| 1:11:30 | 116.34 | 155.12 | 174.12 | 96.28 | 92.4 | 89.2 | 85.08 |
| 1:11:45 | 114.46 | 155.16 | 171.98 | 87.08 | 88.56 | 91.98 | 94.78 |
| 1:12:00 | 114.52 | 154.5 | 173.16 | 94.48 | 92.92 | 90.82 | 84.88 |
| 1:12:15 | 115.64 | 154.54 | 170.02 | 87.1 | 91.98 | 94.5 | 89.4 |
| 1:12:30 | 116.46 | 153.8 | 171.56 | 95.36 | 94.86 | 92.42 | 85.14 |
| 1:12:45 | 114.34 | 153.92 | 167.68 | 87.04 | 90.92 | 93.64 | 90.24 |
| 1:13:00 | 115.08 | 153.36 | 170.8 | 96.22 | 93.1 | 91 | 85.44 |
| 1:13:15 | 115.12 | 153.32 | 165.08 | 87.3 | 91.86 | 93.12 | 90.34 |
| 1:13:30 | 114.76 | 153.02 | 168.98 | 94.6 | 91.22 | 90.52 | 85.5 |
| 1:13:45 | 114.48 | 152.68 | 162.7 | 87.82 | 90 | 89.38 | 90.08 |
| 1:14:00 | 114.86 | 152.64 | 167.02 | 93.08 | 91.28 | 90.66 | 85.58 |
| 1:14:15 | 114.14 | 151.96 | 160.06 | 88.66 | 89.68 | 85.82 | 90.16 |
| 1:14:30 | 114.12 | 152.02 | 165.62 | 93.14 | 90.06 | 89.78 | 85.58 |
| 1:14:45 | 113.92 | 151.44 | 157.3 | 87.38 | 89.12 | 91.4 | 88.86 |
| 1:15:00 | 114.06 | 151.2 | 161.5 | 90.28 | 85.94 | 89.72 | 85.52 |
| 1:15:15 | 113.56 | 151.12 | 154.1 | 87.14 | 88.5 | 87.54 | 89.88 |
| 1:15:30 | 113.9 | 150.94 | 160.8 | 91.4 | 86.54 | 89.46 | 85.5 |
| 1:15:45 | 113.8 | 150.68 | 150.08 | 87.84 | 88.62 | 89.74 | 88.1 |
| 1:16:00 | 112.42 | 149.96 | 153.88 | 90.06 | 85.66 | 89.02 | 85.5 |
| 1:16:15 | 112.94 | 149.76 | 147.38 | 86.74 | 88.3 | 86.06 | 88.06 |
| 1:16:30 | 113.16 | 149.52 | 154.48 | 90.5 | 85.7 | 88.34 | 85.36 |
| 1:16:45 | 113.06 | 148.66 | 144.58 | 86.68 | 88.12 | 88.52 | 87.72 |
| 1:17:00 | 113.18 | 148.2 | 147.48 | 89.88 | 86.36 | 88.3 | 85.26 |
| 1:17:15 | 112.08 | 147.28 | 142.64 | 86.94 | 87.82 | 85.6 | 87.2 |
| 1:17:30 | 111.78 | 146.9 | 147.16 | 89.8 | 85.34 | 88.22 | 85.24 |
| 1:17:45 | 111.66 | 145.84 | 140.72 | 86.5 | 87.34 | 85.74 | 87.8 |
| 1:18:00 | 112.12 | 145.48 | 147.8 | 89.98 | 85.26 | 87.88 | 85.06 |
| 1:18:15 | 112.58 | 144.24 | 136.8 | 86.56 | 87.52 | 85.16 | 87.28 |
| 1:18:30 | 110.84 | 144.58 | 139.84 | 89.66 | 87.22 | 87.7 | 84.94 |
| 1:18:45 | 110.76 | 142.62 | 134.7 | 86.24 | 87.6 | 85.06 | 87 |
| 1:19:00 | 110.38 | 142.5 | 138.48 | 89.08 | 84.92 | 87.4 | 84.86 |
| 1:19:15 | 112.32 | 141.24 | 130.2 | 85.9 | 87.04 | 87.06 | 86.68 |
| 1:19:30 | 111.42 | 143.96 | 127.36 | 88.68 | 84.78 | 86.5 | 84.76 |
| 1:19:45 | 110.28 | 139.92 | 126.66 | 86.9 | 87.22 | 84.98 | 86.1 |
| 1:20:00 | 109.68 | 140.68 | 130.38 | 88.22 | 84.84 | 87.28 | 84.72 |
| 1:20:15 | 110.62 | 139.28 | 123.36 | 85.8 | 87.02 | 85.02 | 86.18 |
| 1:20:30 | 109.76 | 144.54 | 122.38 | 88.18 | 84.62 | 86.6 | 85.06 |
| 1:20:45 | 108.54 | 138.9 | 119.5 | 86.72 | 86.46 | 84.64 | 85.66 |
| 1:21:00 | 108.68 | 142.82 | 120.64 | 88 | 85.3 | 86.54 | 84.44 |
| 1:21:15 | 109.4 | 138.6 | 116.32 | 85.7 | 86.26 | 84.46 | 85.88 |
| 1:21:30 | 107.78 | 140.04 | 120 | 87.7 | 84.3 | 86.34 | 84.44 |
| 1:21:45 | 106.96 | 138.36 | 114.16 | 85.34 | 85.88 | 84.3 | 85.84 |
| 1:22:00 | 106.76 | 143.34 | 115.54 | 87.36 | 84.36 | 86.06 | 84.82 |
| 1:22:15 | 107.6 | 137.88 | 112.26 | 85.38 | 86 | 84.22 | 85.84 |
| 1:22:30 | 106.56 | 142.24 | 111.96 | 87.5 | 84.14 | 85.96 | 84.42 |
| 1:22:45 | 105.42 | 137.56 | 110.66 | 85.12 | 85.94 | 84.18 | 85.02 |
| 1:23:00 | 105.5 | 143.24 | 109.74 | 87.46 | 84.08 | 85.56 | 84.4 |
| 1:23:15 | 105.24 | 137.32 | 109.28 | 84.62 | 85.98 | 84.04 | 85.22 |
| 1:23:30 | 104.18 | 141.24 | 111.96 | 87.02 | 83.94 | 85.98 | 84.42 |
| 1:23:45 | 104.5 | 137.84 | 108.88 | 85.22 | 85.3 | 83.98 | 85.74 |
| 1:24:00 | 103.34 | 135.6 | 109.98 | 86.86 | 83.92 | 85.48 | 83.82 |
| 1:24:15 | 102.98 | 137.32 | 109.38 | 84.9 | 85.48 | 83.94 | 85.24 |
| 1:24:30 | 103.16 | 132.66 | 110.34 | 86.76 | 83.8 | 85.62 | 84.28 |
| 1:24:45 | 102.34 | 135 | 110.14 | 85.36 | 85.44 | 83.76 | 84.76 |
| 1:25:00 | 101.76 | 130.58 | 107.82 | 86.42 | 83.7 | 85.34 | 83.92 |
| 1:25:15 | 101.96 | 132.56 | 109.88 | 85.48 | 85.58 | 83.62 | 84.9 |
| 1:25:30 | 101.24 | 129.56 | 109.4 | 86.4 | 83.6 | 85.28 | 84.16 |
| 1:25:45 | 100.92 | 130.4 | 109.36 | 84.96 | 84.8 | 83.58 | 84.34 |
| 1:26:00 | 100.68 | 127.5 | 108.64 | 85.74 | 83.52 | 85.12 | 84.08 |
| 1:26:15 | 100.4 | 128.94 | 108.88 | 84.66 | 85 | 83.42 | 84.66 |
| 1:26:30 | 99.98 | 126.14 | 108.2 | 86.34 | 83.36 | 85.04 | 84.02 |
| 1:26:45 | 99.86 | 127.42 | 108.12 | 85.28 | 84.92 | 83.4 | 84.76 |
| 1:27:00 | 99.44 | 125.24 | 107.78 | 86.1 | 83.28 | 85.12 | 83.68 |
| 1:27:15 | 99.14 | 125.56 | 107.46 | 84.48 | 84.56 | 83.28 | 84.44 |
| 1:27:30 | 98.86 | 122.32 | 107.52 | 85.92 | 83.18 | 84.74 | 83.84 |
| 1:27:45 | 98.54 | 124.12 | 107.26 | 84.28 | 84.4 | 83.24 | 84.38 |
| 1:28:00 | 98.28 | 121.9 | 107.14 | 85.88 | 83.14 | 84.58 | 83.74 |
| 1:28:15 | 98.18 | 122.82 | 107.08 | 84.52 | 84.76 | 83.16 | 84.68 |
| 1:28:30 | 97.66 | 119.4 | 106.04 | 85.78 | 83.06 | 84.42 | 83.72 |
| 1:28:45 | 97.58 | 121.54 | 106.68 | 84.12 | 84.5 | 83.06 | 83.9 |
| 1:29:00 | 97.26 | 117.52 | 104.86 | 85.58 | 82.98 | 84.3 | 83.68 |
| 1:29:15 | 96.94 | 119.92 | 105.56 | 84.2 | 84.24 | 82.92 | 84.26 |
| 1:29:30 | 96.72 | 117.04 | 102.86 | 85.54 | 82.9 | 84.78 | 83.22 |
| 1:29:45 | 96.76 | 118.16 | 104 | 84.52 | 84.46 | 82.9 | 84.04 |
| 1:30:00 | 96.16 | 114.88 | 100.48 | 85.36 | 82.86 | 84.26 | 83.48 |
| 1:30:15 | 96.22 | 116.64 | 102.74 | 83.9 | 84 | 82.84 | 83.9 |
| 1:30:30 | 96 | 114.62 | 102.16 | 85.92 | 82.76 | 84.66 | 83.1 |
| 1:30:45 | 95.66 | 115.2 | 102.38 | 83.84 | 83.62 | 82.72 | 84.56 |
| 1:31:00 | 95.4 | 113.78 | 102.12 | 85.68 | 82.7 | 84.06 | 83.04 |
| 1:31:15 | 95.48 | 113.94 | 102.44 | 84.12 | 84.28 | 82.64 | 83.56 |
| 1:31:30 | 94.98 | 113.12 | 101.86 | 85.2 | 82.6 | 83.64 | 82.76 |
| 1:31:45 | 94.8 | 112.78 | 102.18 | 84.16 | 84.5 | 82.6 | 83.74 |
| 1:32:00 | 94.8 | 111.82 | 101.54 | 85.52 | 82.52 | 84.7 | 82.9 |
| 1:32:15 | 94.42 | 111.4 | 101.66 | 84.82 | 84.88 | 82.52 | 83.98 |
| 1:32:30 | 94.1 | 110.4 | 101.36 | 85.3 | 82.48 | 83.82 | 83.34 |
| 1:32:45 | 93.92 | 109.86 | 101.28 | 84.1 | 84.86 | 82.44 | 83.24 |
| 1:33:00 | 93.78 | 109.2 | 101.36 | 85.24 | 82.42 | 84.7 | 83.14 |
| 1:33:15 | 93.46 | 108.46 | 101.06 | 83.82 | 83.74 | 82.38 | 83.32 |
| 1:33:30 | 93.14 | 108.16 | 101.16 | 85.34 | 82.36 | 84.06 | 82.82 |
| 1:33:45 | 93.08 | 107.12 | 100.9 | 84.44 | 84.06 | 82.32 | 83.72 |
| 1:34:00 | 92.98 | 106.6 | 100.86 | 84.94 | 82.28 | 83.38 | 82.76 |
| 1:34:15 | 92.9 | 106.06 | 100.76 | 84.26 | 84 | 82.26 | 83.7 |
| 1:34:30 | 92.54 | 105.06 | 100.5 | 84.9 | 82.24 | 83.66 | 83.02 |
| 1:34:45 | 92.74 | 104.62 | 100.48 | 84.02 | 84.12 | 82.22 | 83.12 |
| 1:35:00 | 92.76 | 103.8 | 99.74 | 85.3 | 82.18 | 84.86 | 82.92 |
| 1:35:15 | 92.6 | 103.1 | 100.12 | 83.74 | 83.6 | 82.16 | 83.36 |
| 1:35:30 | 92.72 | 102.68 | 99.34 | 84.64 | 82.14 | 84.54 | 82.72 |
| 1:35:45 | 92.8 | 101.76 | 99.52 | 83.7 | 84.46 | 82.1 | 83.1 |
| 1:36:00 | 92.54 | 101.38 | 99.06 | 84.68 | 82.08 | 83.44 | 82.68 |
| 1:36:15 | 92.5 | 100.68 | 99.1 | 83.9 | 83.68 | 82.04 | 83.2 |
| 1:36:30 | 92.38 | 99.92 | 98.84 | 84.46 | 81.98 | 84.02 | 82.74 |
| 1:36:45 | 92.4 | 99.7 | 98.68 | 83.78 | 83.32 | 82 | 82.98 |
| 1:37:00 | 92.24 | 99.58 | 98.42 | 84.74 | 81.94 | 84.46 | 82.76 |
| 1:37:15 | 92.2 | 98.88 | 98.42 | 83.42 | 83.28 | 81.92 | 82.96 |
| 1:37:30 | 92.1 | 98.42 | 98 | 84.42 | 81.9 | 84.08 | 82.7 |
| 1:37:45 | 92.14 | 98 | 98.12 | 83.74 | 83.68 | 81.88 | 82.98 |
| 1:38:00 | 91.98 | 97.84 | 97.72 | 84.58 | 81.84 | 83.6 | 82.98 |
| 1:38:15 | 91.82 | 97.32 | 97.8 | 83.6 | 83.3 | 81.84 | 83.04 |
| 1:38:30 | 91.7 | 97.02 | 97.66 | 85.3 | 81.8 | 83.22 | 82.54 |
| 1:38:45 | 91.84 | 96.5 | 97.46 | 83.5 | 82.72 | 81.78 | 82.56 |
| 1:39:00 | 91.56 | 96.44 | 97.62 | 84.3 | 81.72 | 83.4 | 82.56 |
| 1:39:15 | 91.7 | 96.16 | 97.28 | 83.8 | 83.34 | 81.7 | 83 |
| 1:39:30 | 91.78 | 95.72 | 97.42 | 84.26 | 81.7 | 83.18 | 82.18 |
| 1:39:45 | 91.4 | 95.82 | 97.26 | 83.7 | 83.94 | 81.68 | 82.78 |
| 1:40:00 | 91.38 | 95.42 | 96.68 | 83.96 | 81.64 | 83.34 | 82.62 |
| 1:40:15 | 91.82 | 95.28 | 97.1 | 83.66 | 83.34 | 81.62 | 82.68 |
| 1:40:30 | 91.84 | 94.58 | 96.14 | 83.86 | 81.58 | 83.38 | 82.54 |
| 1:40:45 | 91.4 | 94.94 | 96.6 | 83.1 | 83.28 | 81.58 | 82.76 |
| 1:41:00 | 91.04 | 93.92 | 95.82 | 84.34 | 81.54 | 82.9 | 82.58 |

1. A method for pasteurizing or sterilizing an architectural coating composition comprising the steps of
(i) providing or optionally preparing the architectural coating composition;
(ii) applying heat from a heat source to said architectural coating composition to pasteurize or sterilize same by heating said architectural coating composition to an internal temperature range from about 100° C. to about 131° C. and dynamically moving said architectural composition through the heat source for any time duration;
(iii) storing the architectural coating composition in containers.

2. The method for pasteurizing or sterilizing the architectural coating composition of claim 1, wherein one of the Stormer or ICI viscosity measurements changes from preheated to post-heated is less than 10%.

3. The method for pasteurizing or sterilizing the architectural coating composition of claim 1, wherein the time duration is from about 1 minute or less to about 5 minutes or less.

4. The method for pasteurizing or sterilizing the architectural coating composition of claim 1, wherein the time duration is about 15 seconds or less.

5. The method for pasteurizing or sterilizing the architectural coating composition of claim 1, wherein step (ii) comprises a continuous heating process.

6. The method for pasteurizing or sterilizing the architectural coating composition of claim 1 further comprising the step of cooling the architectural composition, which occurs after step (ii) and optionally before step (iii).

7. The method for pasteurizing or sterilizing the architectural coating composition of claim 5, wherein the architectural coating composition flows through a continuous piping through the heat source.

8. The method for pasteurizing or sterilizing the architectural coating composition of claim 7, wherein the continuous piping comprises heat transferring fins.

9. The method for pasteurizing of sterilizing of claim 1, wherein step (iii) occurs prior to step (ii).

10. A method for pasteurizing an architectural coating composition comprising the steps of
(i) providing or optionally preparing the architectural coating composition;
(ii) storing the pasteurized architectural coating composition in containers;
(iii) applying heat to said architectural coating composition to pasteurize same by heating said architectural coating composition to an internal temperature range from about 60° C. to about 92.5° C. and dynamically moving said architectural composition through the heat source for a time duration range from at least about 50 minutes to at least about 2 minutes.

11. The method of claim 10, further comprising a step of cooling the architectural coating composition after the applying heat step.

12. The method of claim 10, wherein in the applying heat step a rotary sterilizer-cooler is applying heat to the architectural coating composition.

13. The method of claim 10, wherein the internal temperature range in step (iii) may increase or decrease by any increments of 2.5° C. between the low end and the high end.

14. The method of claim 10, wherein the time duration range in step (iii) may decrease or increase by any increments of 2.5 minutes between the longer end and the shorter end.

15. The method for pasteurizing or sterilizing the architectural coating composition of claim 4, wherein the time duration is 10 seconds or less.

16. The method for pasteurizing or sterilizing the architectural coating composition of claim 15, wherein the time duration is 5 seconds or less.

17. The method for pasteurizing or sterilizing the architectural coating composition of claim 16, wherein the time duration is 2.5 seconds or less.

18. A method for pasteurizing or sterilizing the architectural coating composition comprising the steps of
(i) providing or preparing the architectural coating composition; and
(ii) applying heat to said architectural coating composition to pasteurize same by heating said architectural coating composition to an internal temperature range from about 60° C. to about 92.5° C. and dynamically moving said architectural composition through the heat source for any time duration;
wherein a change in either a Stormer viscosity or ICI viscosity from untreated to post-heat treatment is less than about 10%;
wherein the time duration is about 15 seconds or less.

19. The method for pasteurizing or sterilizing the architectural coating composition of claim 18 further comprising the step of (iii) storing the architectural coating in containers.

20. The method for pasteurizing or sterilizing the architectural coating composition of claim 18, wherein the time duration is about 10 seconds or less.

21. The method for pasteurizing or sterilizing the architectural coating composition of claim 20, wherein the time duration is about 5 seconds or less.

22. The method for pasteurizing or sterilizing the architectural coating composition of claim 21, wherein the time duration is about 2.5 seconds or less.
